(19) United States
(12) Patent Application Publication
(10) Pub. No.: US 2021/0002649 A1
(43) Pub. Date: Jan. 7, 2021

(54) COMPOSITIONS AND METHODS FOR TREATING CANCER

(71) Applicant: Beth Israel Deaconess Medical Center, Inc., Boston, MA (US)

(72) Inventors: Wenyi Wei, Boston, MA (US); Pier Paolo Pandolfi, Boston, MA (US); Yu Ru Lee, Boston, MA (US)

(73) Assignee: Beth Israel Deaconess Medical Center, Inc., Boston, MA (US)

(21) Appl. No.: 16/976,996

(22) PCT Filed: Feb. 28, 2019
(86) PCT No.: PCT/US2019/020101

§ 371 Date: Aug. 31, 2020

Related U.S. Application Data
(60) Provisional application No. 62/637,334, filed on Mar. 1, 2018.

(51) Int. Cl. C12N 15/113; C07K 16/28; A61K 31/404; A61K 31/495; A61P 35/00

(52) U.S. Cl. CPC C12N 15/1137; C07K 16/2818; C07K 16/2827; C12Y 203/02; A61K 31/495; A61P 35/00; A61K 31/404

(57) ABSTRACT

The invention generally provides a method of treating cancer, the method comprising administering to a subject having a cancer, an effective amount of an agent that inhibits the expression or activity of WW domain-containing protein-1 (WWP1) in combination with anti-PD-1 and/or anti-PD-L1 monoclonal antibodies.

### CROSS-REFERENCE TO RELATED APPLICATIONS

This application claims the benefit of and priority to U.S. Provisional Patent Application Ser. No. 62/637,334, filed Mar. 1, 2018, which is incorporated herein by reference in its entirety.

### STATEMENT OF RIGHTS TO INVENTIONS MADE UNDER FEDERALLY SPONSORED RESEARCH

This invention was made with government support under Grant No. CA082328 awarded by the National Institutes of Health. The government has certain rights in the invention.

### BACKGROUND OF THE INVENTION

The Programmed Cell Death Protein 1 (also known as CD279 and PD-1) and its ligand PD-1 Ligand (PD-L1) signaling pathway provide an important immune checkpoint that protects against autoimmunity. Evidence suggests that PD-1 signaling is used by tumors to escape antitumor immune responses. Anti-PD-1 and anti-PD-L1 monoclonal antibodies are useful for treating patients with a variety of cancers. Unfortunately, only a minority of treated patients respond to the current immunotherapy treatment. Methods for improving the efficacy of such therapies is urgently required.

### SUMMARY OF THE INVENTION

The invention generally provides a method of treating cancer, the method comprising administering to a subject having a cancer, an effective amount of an agent that inhibits the expression or activity of WW domain-containing protein-1 (WWP1) in combination with anti-PD-1 and/or anti-PD-L1 monoclonal antibodies.

In some aspects, a method is provided for inhibiting the survival or proliferation of a neoplastic cell, the method comprising contacting the cell with an effective amount of an agent that inhibits the expression or activity of WWP1 and an anti-PD-1 and/or anti-PD-L1 antibody, thereby inhibiting the survival or proliferation of the neoplastic cell. In some embodiments, the neoplastic cell is a mammalian cell. In some embodiments, the mammalian cell is a murine, rat, or human cell. In some embodiments, the cell is in vitro or in vivo. In some embodiments, the neoplastic cell is a prostate cancer, breast cancer, or colorectal cancer cell. In some embodiments,

In some embodiments of the invention disclosed herein, the method also includes administering to the subject or the cell a NEDD4-1 inhibitor. In some embodiments, the subject has prostate cancer, breast cancer, or colorectal cancer. In some embodiments, the agent is a polypeptide, polynucleotide, or a small molecule. In some embodiments, the polynucleotide is an inhibitory nucleic acid molecule that inhibits the expression of WWP1. In some embodiments, the inhibitory nucleic acid molecule is an antisense molecule, siRNA, or shRNA. In other embodiments, the agent is selected from the group consisting of: 4-(4-chlorobenzoyl) piperazin-1-yl) (4-(phonoxymethyl) phenyl) methanone, and indole-3-carbinol.

The details of one or more embodiments of the disclosure are set forth in the description below. Other features or advantages of the present disclosure will be apparent from the following drawings and detailed description of several embodiments, and also from the appending claims.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “Neural Precursor Cell Expressed Developmentally Down-Regulated Protein 4 polypeptide (NEDD4)” or “(NEDD4-1)” is meant a protein having at least about 85% amino acid sequence identity to NCBI Reference Sequence: NP 001271267 and having E3 ubiquitin-protein ligase activity. NEDD4-1 is frequently overexpressed in cancers, such as, for example, gastric adenocarcinoma, colon adenocarcinoma, prostate cancer, bladder cancer, and breast cancer. An exemplary NEDD4-1 amino acid sequence is provided herein below (SEQ ID NO: 3):

| | |
|---|---|
| 1 | matcavevfg lledeensri vrvrviagig lakkdilgas dpyyrytlyd pmngyltsyq |
| 61 | tktikkslnp kwneeilfry hpqqhrllfe vfdenrltrd dflgqvdvpl yplptenprl |
| 121 | erpytfkdfv lhprshksry kgylrlkmty 1pktsgsedd naeqaeelep gwvvldqpda |
| 181 | achlqqqqep splppgweer qdilgrtyyv nhesrrtqwk rptpqdnitd aengniqlqa |
| 241 | grafttrrqi seetesvdnr essenweiir edeatmysnq afpspppssn ldvpthlaee |
| 301 | lnarltifgn saysgpasss nhssrrgslq aytfeeqptl pvllptssgl ppgweekqde |
| 361 | rgrsyyvdhn srtttwtkpt vqatvetsql tssqssagpq sqastsdsgq qvtqpseieg |
| 421 | gflpkgwevr hapngrpffi dhntktttwe dprlkipahl rgktsldtsn dlgplppgwe |
| 481 | erthtdgrif yinhnikrtq wedprlenva itgpavpysr dykrkyeffr rklkkqndip |
| 541 | nkfemklrra tvledsyrri mgvkradflk arlwiefdge kgldyggvar ewffliskem |
| 601 | fnpyyglfey satdnytlqi npnsglcned hlsyfkfigr vagmavyhgk lldgffirpf |
| 661 | ykmmlhkpit lhdmesvdse yynslrwile ndpteldlrf iideelfgqt hqhelknggs |
| 721 | eivvtnknkk eyiylviqwr fvnriqkqma afkegffeli pqdlikifde nelellmcgl |
| 781 | gdvdvndwre htkykngysa nhqviqwfwk avlmmdsekr irllqfvtgt srvpmngfae |
| 841 | lygsngpqsf tveqwgtpek 1prahtcfnr ldlppyesfe elwdklqmai entqgfdgvd |

By“NEDD4 or NEDD4-1 polynucleotide” is meant a nucleic acid molecule encoding a NEDD4-1 polypeptide. An exemplary NEDD4-1 polynucleotide sequence is provided at NCBI Reference Sequence: NM 001284338, and reproduced herein below (SEQ ID NO: 4):

| | |
|---|---|
| 1 | gctgcagctg agctcagctt aaaggtcgct ggaaatcaag tgctttgtaa gtgaaggcat |
| 61 | tttgactaca agcgattcaa gacattttgg aaacgtcagt ctctggaatg ggtcgtcttt |
| 121 | ttctttctat gatatctggt atctaatgtt ttacttagga taaagactac tacttaatag |
| 181 | ctcttttagg caccacaaat gattttttct tttgatgcag tgcagctgct tctagcctgt |
| 241 | ctacggacag agatctttgt tatgactttc agcccttaac atgtttggaa atgagaacaa |
| 301 | atggcacaaa gcttacgatt gcactttgca gccagaagaa gcaatactta ccctttgtca |
| 361 | gaaacctccg gagatgactt ggatagccat gttcacatgt gcttcaaaag accaacacgg |
| 421 | atttcaacgt ctaacgttgt tcaaatgaag ctgactccca gacagactgc actagctccg |
| 481 | ttaataaagg aaaacgttca gtctcaagaa agatcatctg ttccctcatc tgaaaatgtt |
| 541 | aataaaaaga gcagctgtct acagatttca ctacagccaa caaggtacag tggatatctt |
| 601 | cagtctagca atgtcttagc tgatagtgat gatgcttcgt ttacttgtat cttgaaggat |
| 661 | ggtatttaca gtagtgctgt ggtcgataat gaattgaatg ctgtgaatga tggtcacctt |
| 721 | gtaagcagtc cagccatttg tagtggtagc cttagtaact tttcaaccag tgataatggg |
| 781 | tcttacagca gcaacggtag tgattttggg tcatgtgcaa gtatcacaag tggaggttca |
| 841 | tatactaaca gtgtcatcag tgacagtagt agttatactt ttccaccaag tgatgatact |
| 901 | tttttgggtg gaaacttacc ttctgacagc acctccaata gaagtgtgcc aaacaggaat |
| 961 | actactcctt gtgaaatttt ttcaagaagt acaagtacag atccttttgt ccaggatgac |
| 1021 | ttggaacatg gattagagat tatgaaattg ccagtgagca ggaacacaaa aattccacta |
| 1081 | aaacgttact cctccttagt catttttcct aggagtcctt caactacccg accgacttct |
| 1141 | ccaacaagtc tgtgtactct tctgagcaaa ggatcctatc aaacttcaca ccagtttatt |
| 1201 | atttctccta gtgaaattgc acataatgag gatggcacta gtgctaaagg atttctttca |
| 1261 | acagctgtca atggacttcg gttatctaaa acaatttgta cccccggaga agtaagagac |
| 1321 | atacggccgc ttcacaggaa gggctcgtta cagaagaaaa ttgttctttc gaataatact |
| 1381 | cccagacaga ctgtctgtga aaagtcatct gaaggatatt cttgtgtttc agtgcatttc |
| 1441 | acccaacgaa aagcagctac attagactgt gaaacaacaa atggtgattg taaaccagaa |
| 1501 | atgtcagaaa ttaagcttaa ttctgattca gagtatatta agctcatgca taggacatct |
| 1561 | gcatgtttgc catcctccca aaatgtagat tgtcaaataa atatcaatgg agaattggaa |
| 1621 | agaccacatt cacagatgaa caaaaaccat ggtattttac gaagaagtat ttcattggga |
| 1681 | ggagcttatc caaatatttc ctgtctatcc agccttaagc acaattgttc taaaggggga |
| 1741 | ccatctcagt tactcataaa gtttgcatct ggaaatgaag gtaaagtgga taatttatca |
| 1801 | agagacagca acagagattg cacaaatgaa ctgtctaatt cttgcaagac aagagatgat |
| 1861 | ttcctaggtc aagtggatgt tccactttat ccattaccga cagaaaatcc aagattggag |
| 1921 | agaccatata catttaagga ttttgttctt catccaagaa gtcacaaatc aagagttaaa |
| 1981 | ggttatctga gactaaaaat gacttattta cctaaaacca gtggctcaga agatgataat |
| 2041 | gcagaacagg ctgaggaatt agagcctggc tgggttgttt tggaccaacc agatgctgct |
| 2101 | tgccatttgc agcaacaaca agaaccttct cctctacctc cagggtggga agagaggcag |
| 2161 | gatatccttg gaaggaccta ttatgtaaac catgaatcta gaagaacaca gtggaaaaga |
| 2221 | ccaacccctc aggacaacct aacagatgct gagaatggca acattcaact gcaagcacaa |
| 2281 | cgtgcattta ccaccaggcg gcagatatcc gaggaaacag aaagtgttga caaccgagag |
| 2341 | tcttccgaga actgggaaat tataagagaa gatgaagcca ccatgtatag caaccaggcc |
| 2401 | ttcccatcac ctccaccgtc aagtaacttg gatgttccaa ctcatcttgc agaagaattg |
| 2461 | aatgccagac tcaccatttt tggaaattca gccgtgagcc agccagcatc gagctcaaat |
| 2521 | cattccagca gaagaggcag cttacaagcc tatacttttg aggaacaacc tacacttcct |
| 2581 | gtgcttttgc ctacttcatc tggattacca ccaggttggg aagaaaaaca agatgaaaga |
| 2641 | ggaagatcat attatgtaga tcacaattcc agaacgacta cttggacaaa gcccactgta |
| 2701 | caggccacag tggagaccag tcagctgacc tcaagccaga gttctgcagg ccctcaatca |
| 2761 | caagcctcca ccagtgattc aggccagcag gtgacccagc catctgaaat tgagcaagga |
| 2821 | ttccttccta aaggctggga agtccggcat gcaccaaatg ggaggccttt ctttattgac |
| 2881 | cacaacacta aaaccaccac ctgggaagat ccaagattga aaattccagc ccatctgaga |
| 2941 | ggaaagacat cacttgatac ttccaatgat ctagggcctt tacctccagg atgggaagag |
| 3001 | agaactcaca cagatggaag aatcttctac ataaatcaca atataaaaag aacacaatgg |
| 3061 | gaagatcctc ggttggagaa tgtagcaata actggaccag cagtgcccta ctccagggat |
| 3121 | tacaaaagaa agtatgagtt cttccgaaga aagttgaaga agcagaatga cattccaaac |
| 3181 | aaatttgaaa tgaaacttcg ccgagcaact gttcttgaag actcttaccg gagaattatg |
| 3241 | ggtgtcaaga gagcagactt cctgaaggct cgactgtgga ttgagtttga tggtgaaaag |
| 3301 | ggattggatt atggaggagt tgccagagaa tggttcttcc tgatctcaaa ggaaatgttt |
| 3361 | aacccttatt atgggttgtt tgaatattct gctacggaca attataccct acagataaat |
| 3421 | ccaaactctg gattgtgtaa cgaagatcac ctctcttact tcaagtttat tggtcgggta |
| 3481 | gctggaatgg cagtttatca tggcaaactg ttggatggtt ttttcatccg cccattttac |
| 3541 | aagatgatgc ttcacaaacc aataaccctt catgatatgg aatctgtgga tagtgaatat |
| 3601 | tacaattccc taagatggat tcttgaaaat gacccaacag aattggacct caggtttatc |
| 3661 | atagatgaag aactttttgg acagacacat caacatgagc tgaaaaatgg tggatcagaa |
| 3721 | atagttgtca ccaataagaa caaaaaggaa tatatttatc ttgtaataca atggcgattt |
| 3781 | gtaaaccgaa tccagaagca aatggctgct tttaaagagg gattctttga actaatacca |
| 3841 | caggatctca tcaaaatttt tgatgaaaat gaactagagc ttcttatgtg tggactggga |
| 3901 | gatgttgatg tgaatgactg gagggaacat acaaagtata aaaatggcta cagtgcaaat |
| 3961 | catcaggtta tacagtggtt ttggaaggct gttttaatga tggattcaga aaaaagaata |
| 4021 | agattacttc agtttgtcac tggcacatct cgggtgccta tgaatggatt tgctgaacta |
| 4081 | tacggttcaa atggaccaca gtcatttaca gttgaacagt ggggtactcc tgaaaagctg |
| 4141 | ccaagagctc atacctgttt taatcgcctg gacttgccac cttatgaatc atttgaagaa |
| 4201 | ttatgggata aacttcagat ggcaattgaa aacacccagg gctttgatgg agttgattag |
| 4261 | attacaaata acaatctgta gtgtttttac tgccatagtt ttataaccaa aatcttgact |
| 4321 | taaaattttc cggggaacta ctaaaatgtg gccactgagt cttcccagat cttgaagaaa |
| 4381 | atcatataaa aagcatttga agaaatagta cgacaactta tttttaatca cttttaaata |
| 4441 | atgtgttgca tttacacagt tgtttcatgc tgtctttaga gttaggtgcc tgcctaaagc |
| 4501 | caggcaccac cacacctggc tttagagttc acacaatagg atataagtcc tgtatgactt |
| 4561 | aaatagtgaa ttttgtcctt aacatttacc tcttgtatag tatctgccag gcagtttttt |
| 4621 | cttaaactac tgagatgata actgtgaaat atttgtgata cgtgtcatgt gtgaaaagtt |
| 4681 | tgatgcattt tgagatggaa aactgaaatt tggaaaaaga aatactttac tattgagtaa |
| 4741 | actacaatat atttagtgct actcgcagct atttattatt ttgtagacct gccttatgca |
| 4801 | ccttactgcc tagatttttg ggaaaaaact ttggaaagtg tgttacctat atttctagcc |
| 4861 | aactaactca cagaaaaact gtttacttct tcactttcga agtatttggc ttttgttaat |
| 4921 | atgcagtttt actaaacaga tggttcataa gacatgtgaa gcaaattcat atttgcaatg |
| 4981 | ggtaaaaagt attaaagcct ttctcttgcc tgcatatcct attgaccatt ggtatagtca |
| 5041 | tcactttttc atattttagt gtagttagaa gaattccttc ttcaaacatt aaagatccac |
| 5101 | aaagcagtat ttctaaatat gccttgaaga actaaatgaa gtgtatagca cttgccttta |
| 5161 | ctagatattc tttacacttg tacaattatg tagtaaatgt atgtttacag ggtttatcat |
| 5221 | gtttacagat taagctaatt tctgtagtcg catttttata tttttagtat cactctagta |
| 5281 | aaaaaaccaa ataatttgtt taaaataacc aaagagttgt tataatgcat aatttgtatt |
| 5341 | aaatttatta ctatttctta tgccttttaa aatactgttt actatgaaga caatgttttt |
| 5401 | aattacaaat ccagaattct gtaggcaaaa tgctacagtt catatcttcc tttaaccaaa |
| 5461 | ctgaagtaca taaagaccat gtacatgtat tcatcaaacg tttattgaat gctgcgtgcc |
| 5521 | tagcgctgtg ctatgctctg gggtaagagt tgtcagcttc agagaagctg agtcctgatc |
| 5581 | ctcaaggaac ttgcaaatgt gtctatgaat ttgtaaaaca atcaaaagta ggcgtaagca |
| 5641 | gaataaggca aaagggaaag tgttctaggt tccagcacac ctgcaaagat aaagtgtgcc |
| 5701 | aagactgtat ttatatttca tacttatatt gtttcatcct tatattggaa tgattatata |
| 5761 | gaaaatgctc ttaaaaagat taaacctatt tctcagtatg gtatcttggt gatttaggaa |
| 5821 | taattgtaaa tatatgttac gaatcttctt aaatatatat atatacacac cctgtgagaa |
| 5881 | ctgtaaaaag tacctctggt tcttggttta agtttgttgg ggtataacat gatgagtact |
| 5941 | cattagcacc tgatagaaat ctgaaatgtg acagtagcaa aaccactttc tactttccaa |
| 6001 | acaccacagc atcagcatgg tttaggggaa gcaattcaca gattaatgta ccctgcgttt |
| 6061 | tgtcttcacc attgtcaacc agcagtcaag gatgagcacc aggtatagtc ctggtttgat |
| 6121 | atctcaccag ccatgtgaac tgaagcagct tacttaacct ctctggattc atatttcttc |
| 6181 | atctgtcaaa tgagagtaat catgcccacc ctcattgtca tgccagattg tcagaagttc |
| 6241 | cagacagatg agaaagcaaa aagtactttg taaagtgtta tacaatcaga aggtgctttg |
| 6301 | tgctgaccgt cagattgtgt caagtcaggt gtgcaaattg accacactcc caaagaatca |
| 6361 | tttaaaagta aagtgacttc taagagacaa ggaagtagga acatttctga tattaaatat |
| 6421 | ataaataata tggatacaca tgtgtatatc tgtatatgga tcttaccatc ataattcatt |
| 6481 | ttcttcatat cagcaaagta atagtaatgt gggccaatgc attttggaaa tgtcctcatt |
| 6541 | atgtagaatg gaatgtgaaa attatttttg ttaaaacagg attttgccac aattatttaa |
| 6601 | atattatgtt tgtgaactat ctaagcatga gaaaatacaa agctttcttt gtatctggct |
| 6661 | gatactcatt tggatgattc tgtgactcat agaacatgat gttaaatgag accagactca |
| 6721 | actgccacca gtccccagct gcagaccttc atccccttca tctcccacta ggggctcgtg |
| 6781 | gtctggaaga aacatctatc aagcaacaca gccccttatc ccagatagaa aagtgtctca |
| 6841 | aatgcattct actgcgggac agtcagtagg atcattttca taaagcaagg acatcatatg |
| 6901 | tttctagaaa ttacaagcat ataactttag cctacaatct cttattaaaa atttttaaca |
| 6961 | aaattgtatt acaaatgcat ttcatcagaa ctcaaattta aatggtgttt gttttgggtt |
| 7021 | tttatttata atgctgaagt tattccatat aagtatcaag ttaaacacaa ttcattttga |
| 7081 | ttataaacta tttgactttt taaaatcttc tgacacagta aatatatata tcaagattga |
| 7141 | tgtatcaaaa tttattgcac actttaaagt gtaaaatcat tttttaaaat cttgaatcca |
| 7201 | caaataaagt tctattctga ttttaaaaaa caaaacaaaa |

By “Phosphatase And Tensin Homolog Deleted On Chromosome Ten (PTEN) polypeptide” is meant a protein having at least about 85% amino acid identity to the sequence provided at NCBI Reference Sequence: NP 002985.1, or a fragment thereof, and having phosphatase activity. Examples of PTEN proteins include the human PTEN protein having the sequence listed in the NCBI reference sequence NP 000305.3, the sequence of which is provided herein below (SEQ ID NO: 1):

| | |
|---|---|
| 1 | mtaiikeivs rnkrryqedg fdldltyiyp niiamgfpae rlegvyrnni ddvvrfldsk |
| 61 | hknhykiynl caerhydtak fncrvaqypf edhnppqlel ikpfcedldq wlseddnhva |
| 121 | aihckagkgr tgvmicayll hrgkflkaqe aldfygevrt rdkkgvtips qrryvyyysy |
| 181 | llknhldyrp vallfhkmmf etipmfsggt cnpqfvvcql kvkiyssnsg ptrredkfmy |
| 241 | fefpqplpvc gdikveffhk qnkmlkkdkm fhfwvntffi pgpeetsekv engslcdgei |
| 301 | dsicsierad ndkeylv1t1 tkndldkank dkanryfspn fkvklyftkt veepsnpeas |
| 361 | sstsvtpdvs dnepdhyrys dttdsdpene pfdedghtqi tkv |

By “PTEN polynucleotide” is meant a nucleic acid molecule encoding a PTEN polypeptide. An exemplary PTEN polynucleotide sequence is provided at NCBI Reference Sequence: NM_000314.6, and reproduced herein below (SEQ ID NO: 2).

| | |
|---|---|
| 1 | cctcccctcg cccggcgcgg tcccgtccgc ctctcgctcg cctcccgcct cccctcggtc |
| 61 | ttccgaggcg cccgggctcc cggcgcggcg gcggaggggg cgggcaggcc ggcgggcggt |
| 121 | gatgtggcgg gactctttat gcgctgcggc aggatacgcg ctcggcgctg ggacgcgact |
| 181 | gcgctcagtt ctctcctctc ggaagctgca gccatgatgg aagtttgaga gttgagccgc |
| 241 | tgtgaggcga ggccgggctc aggcgaggga gatgagagac ggcggcggcc gcggcccgga |
| 301 | gcccctctca gcgcctgtga gcagccgcgg gggcagcgcc ctcggggagc cggccggcct |
| 361 | gcggcggcgg cagcggcggc gtttctcgcc tcctcttcgt cttttctaac cgtgcagcct |
| 421 | cttcctcggc ttctcctgaa agggaaggtg gaagccgtgg gctcgggcgg gagccggctg |
| 481 | aggcgcggcg gcggcggcgg cacctcccgc tcctggagcg ggggggagaa gcggcggcgg |
| 541 | cggcggccgc ggcggctgca gctccaggga gggggtctga gtcgcctgtc accatttcca |
| 601 | gggctgggaa cgccggagag ttggtctctc cccttctact gcctccaaca cggcggcggc |
| 661 | ggcggcggca catccaggga cccgggccgg ttttaaacct cccgtccgcc gccgccgcac |
| 721 | cccccgtggc ccgggctccg gaggccgccg gcggaggcag ccgttcggag gattattcgt |
| 781 | cttctcccca ttccgctgcc gccgctgcca ggcctctggc tgctgaggag aagcaggccc |
| 841 | agtcgctgca accatccagc agccgccgca gcagccatta cccggctgcg gtccagagcc |
| 901 | aagcggcggc agagcgaggg gcatcagcta ccgccaagtc cagagccatt tccatcctgc |
| 961 | agaagaagcc ccgccaccag cagcttctgc catctctctc ctcctttttc ttcagccaca |
| 1021 | ggctcccaga catgacagcc atcatcaaag agatcgttag cagaaacaaa aggagatatc |
| 1081 | aagaggatgg attcgactta gacttgacct atatttatcc aaacattatt gctatgggat |
| 1141 | ttcctgcaga aagacttgaa ggcgtataca ggaacaatat tgatgatgta gtaaggtttt |
| 1201 | tggattcaaa gcataaaaac cattacaaga tatacaatct ttgtgctgaa agacattatg |
| 1261 | acaccgccaa atttaattgc agagttgcac aatatccttt tgaagaccat aacccaccac |
| 1321 | agctagaact tatcaaaccc ttttgtgaag atcttgacca atggctaagt gaagatgaca |
| 1381 | atcatgttgc agcaattcac tgtaaagctg gaaagggacg aactggtgta atgatatgtg |
| 1441 | catatttatt acatcggggc aaatttttaa aggcacaaga ggccctagat ttctatgggg |
| 1501 | aagtaaggac cagagacaaa aagggagtaa ctattcccag tcagaggcgc tatgtgtatt |
| 1561 | attatagcta cctgttaaag aatcatctgg attatagacc agtggcactg ttgtttcaca |
| 1621 | agatgatgtt tgaaactatt ccaatgttca gtggcggaac ttgcaatcct cagtttgtgg |
| 1681 | tctgccagct aaaggtgaag atatattcct ccaattcagg acccacacga cgggaagaca |
| 1741 | agttcatgta ctttgagttc cctcagccgt tacctgtgtg tggtgatatc aaagtagagt |
| 1801 | tcttccacaa acagaacaag atgctaaaaa aggacaaaat gtttcacttt tgggtaaata |
| 1861 | cattcttcat accaggacca gaggaaacct cagaaaaagt agaaaatgga agtctatgtg |
| 1921 | atcaagaaat cgatagcatt tgcagtatag agcgtgcaga taatgacaag gaatatctag |
| 1981 | tacttacttt aacaaaaaat gatcttgaca aagcaaataa agacaaagcc aaccgatact |
| 2041 | tttctccaaa ttttaaggtg aagctgtact tcacaaaaac agtagaggag ccgtcaaatc |
| 2101 | cagaggctag cagttcaact tctgtaacac cagatgttag tgacaatgaa cctgatcatt |
| 2161 | atagatattc tgacaccact gactctgatc cagagaatga accttttgat gaagatcagc |
| 2221 | atacacaaat tacaaaagtc tgaatttttt tttatcaaga gggataaaac accatgaaaa |
| 2281 | taaacttgaa taaactgaaa atggaccttt ttttttttaa tggcaatagg acattgtgtc |
| 2341 | agattaccag ttataggaac aattctcttt tcctgaccaa tcttgtttta ccctatacat |
| 2401 | ccacagggtt ttgacacttg ttgtccagtt gaaaaaaggt tgtgtagctg tgtcatgtat |
| 2461 | ataccttttt gtgtcaaaag gacatttaaa attcaattag gattaataaa gatggcactt |
| 2521 | tcccgtttta ttccagtttt ataaaaagtg gagacagact gatgtgtata cgtaggaatt |
| 2581 | ttttcctttt gtgttctgtc accaactgaa gtggctaaag agctttgtga tatactggtt |
| 2641 | cacatcctac ccctttgcac ttgtggcaac agataagttt gcagttggct aagagaggtt |
| 2701 | tccgaagggt tttgctacat tctaatgcat gtattcgggt taggggaatg gagggaatgc |
| 2761 | tcagaaagga aataatttta tgctggactc tggaccatat accatctcca gctatttaca |
| 2821 | cacacctttc tttagcatgc tacagttatt aatctggaca ttcgaggaat tggccgctgt |
| 2881 | cactgcttgt tgtttgcgca ttttttttta aagcatattg gtgctagaaa aggcagctaa |
| 2941 | aggaagtgaa tctgtattgg ggtacaggaa tgaaccttct gcaacatctt aagatccaca |
| 3001 | aatgaaggga tataaaaata atgtcatagg taagaaacac agcaacaatg acttaaccat |
| 3061 | ataaatgtgg aggctatcaa caaagaatgg gcttgaaaca ttataaaaat tgacaatgat |
| 3121 | ttattaaata tgttttctca attgtaacga cttctccatc tcctgtgtaa tcaaggccag |
| 3181 | tgctaaaatt cagatgctgt tagtacctac atcagtcaac aacttacact tattttacta |
| 3241 | gttttcaatc ataatacctg ctgtggatgc ttcatgtgct gcctgcaagc ttcttttttc |
| 3301 | tcattaaata taaaatattt tgtaatgctg cacagaaatt ttcaatttga gattctacag |
| 3361 | taagcgtttt ttttctttga agatttatga tgcacttatt caatagctgt cagccgttcc |
| 3421 | acccttttga ccttacacat tctattacaa tgaattttgc agttttgcac attttttaaa |
| 3481 | tgtcattaac tgttagggaa ttttacttga atactgaata catataatgt ttatattaaa |
| 3541 | aaggacattt gtgttaaaaa ggaaattaga gttgcagtaa actttcaatg ctgcacacaa |
| 3601 | aaaaaagaca tttgattttt cagtagaaat tgtcctacat gtgctttatt gatttgctat |
| 3661 | tgaaagaata gggttttttt tttttttttt tttttttttt ttaaatgtgc agtgttgaat |
| 3721 | catttcttca tagtgctccc ccgagttggg actagggctt caatttcact tcttaaaaaa |
| 3781 | aatcatcata tatttgatat gcccagactg catacgattt taagcggagt acaactacta |
| 3841 | ttgtaaagct aatgtgaaga tattattaaa aaggtttttt tttccagaaa tttggtgtct |
| 3901 | tcaaattata ccttcacctt gacatttgaa tatccagcca ttttgtttct taatggtata |
| 3961 | aaattccatt ttcaataact tattggtgct gaaattgttc actagctgtg gtctgaccta |
| 4021 | gttaatttac aaatacagat tgaataggac ctactagagc agcatttata gagtttgatg |
| 4081 | gcaaatagat taggcagaac ttcatctaaa atattcttag taaataatgt tgacacgttt |
| 4141 | tccatacctt gtcagtttca ttcaacaatt tttaaatttt taacaaagct cttaggattt |
| 4201 | acacatttat atttaaacat tgatatatag agtattgatt gattgctcat aagttaaatt |
| 4261 | ggtaaagtta gagacaacta ttctaacacc tcaccattga aatttatatg ccaccttgtc |
| 4321 | tttcataaaa gctgaaaatt gttacctaaa atgaaaatca acttcatgtt ttgaagatag |
| 4381 | ttataaatat tgttctttgt tacaatttcg ggcaccgcat attaaaacgt aactttattg |
| 4441 | ttccaatatg taacatggag ggccaggtca taaataatga cattataatg ggcttttgca |
| 4501 | ctgttattat ttttcctttg gaatgtgaag gtctgaatga gggttttgat tttgaatgtt |
| 4561 | tcaatgtttt tgagaagcct tgcttacatt ttatggtgta gtcattggaa atggaaaaat |
| 4621 | ggcattatat atattatata tataaatata tattatacat actctcctta ctttatttca |
| 4681 | gttaccatcc ccatagaatt tgacaagaat tgctatgact gaaaggtttt cgagtcctaa |
| 4741 | ttaaaacttt atttatggca gtattcataa ttagcctgaa atgcattctg taggtaatct |
| 4801 | ctgagtttct ggaatatttt cttagacttt ttggatgtgc agcagcttac atgtctgaag |
| 4861 | ttacttgaag gcatcacttt taagaaagct tacagttggg ccctgtacca tcccaagtcc |
| 4921 | tttgtagctc ctcttgaaca tgtttgccat acttttaaaa gggtagttga ataaatagca |
| 4981 | tcaccattct ttgctgtggc acaggttata aacttaagtg gagtttaccg gcagcatcaa |
| 5041 | atgtttcagc tttaaaaaat aaaagtaggg tacaagttta atgtttagtt ctagaaattt |
| 5101 | tgtgcaatat gttcataacg atggctgtgg ttgccacaaa gtgcctcgtt tacctttaaa |
| 5161 | tactgttaat gtgtcatgca tgcagatgga aggggtggaa ctgtgcacta aagtgggggc |
| 5221 | tttaactgta gtatttggca gagttgcctt ctacctgcca gttcaaaagt tcaacctgtt |
| 5281 | ttcatataga atatatatac taaaaaattt cagtctgtta aacagcctta ctctgattca |
| 5341 | gcctcttcag atactcttgt gctgtgcagc agtggctctg tgtgtaaatg ctatgcactg |
| 5401 | aggatacaca aaaataccaa tatgatgtgt acaggataat gcctcatccc aatcagatgt |
| 5461 | ccatttgtta ttgtgtttgt taacaaccct ttatctctta gtgttataaa ctccacttaa |
| 5521 | aactgattaa agtctcattc ttgtcattgt gtgggtgttt tattaaatga gagtttataa |
| 5581 | ttcaaattgc ttaagtccat tgaagtttta attaatgggc agccaaatgt gaatacaaag |
| 5641 | ttttcagttt ttttttttcc tgctgtcctt caaagcctac tgtttaaaaa aaaaaaaaaa |
| 5701 | aaaaaacatg gcctgagagt agagtatctg tctactcatg tttaattaag gaaaaacact |
| 5761 | tatttttagg gctttagtca tcacttcata aattgtataa gcacattaaa tagcgttcta |
| 5821 | gtcctgaaaa agtccaagat tcttagaaaa ttgtgcatat ttttattatg acagatgttt |
| 5881 | gaagataatt ccccagaatg gatttgatac tttagatttc aattttgtgg cttttgtcta |
| 5941 | ttattctgta ctctgccatc agcatatgga aagcttcatt tactcatcat gacttgtgcc |
| 6001 | atataaaaat tgatatttcg gaatagtcta aaggactttt tgtacttgaa tttaatcatg |
| 6061 | ttgtttctaa tattcttaaa agcttgaaga ctaaagcata tcctttcaac aaagcatagt |
| 6121 | aaggtaataa gaaagtgtag tttgtacaag tgttaaaaaa ataaagtaga caatgttaca |
| 6181 | gtgggactta ttatttcaag tttacatttt ctccatgtaa ttttttaaaa agtaaatgaa |
| 6241 | aaaatgtgca ataatgtaaa atatgaagtg tatgtgtaca cacattttat ttttcggtat |
| 6301 | cttgggtata cgtatggttg aaaactatac tggagtctaa aagtattcta atttataaga |
| 6361 | agacattttg gtgatgtttg aaaaatagaa atgtgctagt tttgttttta tatcatgtcc |
| 6421 | tttgtacgtt gtaatatgag ctggcttggt tcagtaaatg ccatcaccat ttccattgag |
| 6481 | aatttaaaac tcaccagtgt ttaatatgca ggcttccaaa ggcttatgaa aaaaatcaag |
| 6541 | acccttaaat ctagttaatt tgctgctaac atgaaactct ttggttcttt tatttttgcc |
| 6601 | agataattag acacacatct aaagcttagt cttaaatggc ttaagtgtag ctattgatta |
| 6661 | gtgctgttgc tagttcagaa agaaatgttt gtgaatggaa acaagaatat tcagtccaaa |
| 6721 | ctgttgtaag gacagtacct gaaaaccagg aaacaggata atggaaaaag tcttttaaag |
| 6781 | atgaaatgtt ggagccaact ttcttataga attaattgta tgtggctata gaaagcctaa |
| 6841 | tgattgttgc ttatttttga gagcatatta ttcttttatg accataatct tgctgttttt |
| 6901 | ccatcttcca aaagatcttc cttctaatat gtatatcaga atgtgggtag ccagtcagac |
| 6961 | aaattcatat tggttggtag ctttaaaaag tttgtaatgt gaagacagga aaggacaaaa |
| 7021 | tagtttgctt tggtggtagt actctggttg ttaagctagg tattttgaga ctacttcccc |
| 7081 | atcacaacaa caataaaata atcactcata atcctatcac ctggagacat agccatcgtt |
| 7141 | aatatgttag tgactataca atcatgtttt cttctgtata tccatgtata ttctttaaaa |
| 7201 | atgaaattta tactgtacct gatctcaaag ctttttagct tagtatatct gtcatgaatt |
| 7261 | tgtaggatgt tccattgcat cagaaaacgg acagtgattt gattactttc taatgccaca |
| 7321 | gatgcagatt acatgtagtt attgagaatc ctttcgaatt cagtggctta atcatgaatg |
| 7381 | tctaaatatt gttgacatta ggatgataca tgtaaattaa agttacattt gtttagcata |
| 7441 | gacaagctta acattgtaga tgtttctctt caaaaatcat cttaaacatt tgcatttgga |
| 7501 | attgtgttaa atagaatgtg tgaaacactg tattagtaaa cttcatcacc tttctacttc |
| 7561 | cttatagttt gaacttttca gtttttgtag ttcccaaaca gttgctcaat ttagagcaaa |
| 7621 | ttaatttaac acctgccaaa aaaaggctgc tgttggctta tcagttgtct ttaaattcaa |
| 7681 | atgctcatgt gacttttatc acatcaaaaa atatttcatt aatgattcac ctttagctct |
| 7741 | gaaaattacc gcgtttagta attatagtgg gcttataaaa acatgcaact ctttttgata |
| 7801 | gttatttgag aattttggtg aaaaatattt agctgagggc agtatagaac ttataaacca |
| 7861 | atatattgat atttttaaaa catttttaca tataagtaaa ctgccatctt tgagcataac |
| 7921 | tacatttaaa aataaagctg catattttta aatcaagtgt ttaacaagaa tttatatttt |
| 7981 | ttatttttta aaattaaaaa taatttatat ttcctctgtt gcatgaggat tctcatctgt |
| 8041 | gcttataatg gttagagatt ttatttgtgt ggaatgaagt gaggcttgta gtcatggttc |
| 8101 | tagtgtttca gtttgccaag tctgtttact gcagtgaaat tcatcaaatg tttcagtgtg |
| 8161 | gttttctgta gcctatcatt tactggctat ttttttatgt acacctttag gattttctgc |
| 8221 | ctactctatc cagttgtcca aatgatatcc tacattttac aaatgccctt tcagtttcta |
| 8281 | ttttcttttt ccattaaatt gccctcatgt cctaatgtgc agtttgtaag tgtgtgtgtg |
| 8341 | tgtgtctgtg tgtgtgtgaa tttgattttc aagagtgcta gacttccaat ttgagagatt |
| 8401 | aaataattta attcaggcaa acatttttca ttggaatttc acagttcatt gtaatgaaaa |
| 8461 | tgttaatcct ggatgacctt tgacatacag taatgaatct tggatattaa tgaatttgtt |
| 8521 | agtagcatct tgatgtgtgt tttaatgagt tattttcaaa gttgtgcatt aaaccaaagt |
| 8581 | tggcatactg gaagtgttta tatcaagttc catttggcta ctgatggaca aaaaatagaa |
| 8641 | atgccttcct atggagagta tttttccttt aaaaaattaa aaaggttaat tattttgact |
| 8701 | aaaaaaaaaa aaaaaaaa |

By “WW domain containing E3 ubiquitin protein ligase 1 (WWP1) polypeptide” is meant a protein having about 85% amino acid sequence identity to NCBI Reference Sequence: NP_008944.1 and having E3 ligase activity. WWP1 is frequently overexpressed in cancers, such as, for example, prostate cancer, breast cancer, gastric carcinoma, and liver cancer. An exemplary WWP1 amino acid sequence is provided herein below (SEQ ID NO: 5):

| | |
|---|---|
| 1 | matasprsdt snnhsgrlql qvtvssaklk rkknwfgtai ytevvvdgei tktaksssss |
| 61 | npkwdeqltv nvtpqttlef qvwshrtlka dallgkatid lkgallihnr klervkeqlk |
| 121 | lslenkngia qtgeltvvld glvieqenit ncsssptiei qengdalhen gepsarttar |
| 181 | lavegtngid nhvptstivq nsccsyvvng dntpsspsqv aarpkntpap kplasepadd |
| 241 | tvngesssfa ptdnasvtgt pvvseenals pnctsttved ppvqeiltss ennecipsts |
| 301 | aelesearsi lepdtsnsrs ssafeaaksr qpdgcmdpvr qqsgnantet 1psgweqrkd |
| 361 | phgrtyyvdh ntrtttwerp qplppgwerr vddrrrvyyv dhntrtttwq rptmesvrnf |
| 421 | eqwqsqrnql qgamqqfnqr ylysasmlaa endpygplpp gwekrvdstd rvyfvnhntk |
| 481 | ttqwedprtq glqneeplpe gweirytreg vryfvdhntr tttfkdprng kssvtkggpq |
| 541 | iayergfrwk lahfrylcqs nalpshvkin vsrqtlfeds fqqimalkpy dlrrrlyvif |
| 601 | rgeegldygg larewfflls hevinpmycl feyagknnyc lqinpastin pdhlsyfcfi |
| 661 | grfiamalfh gkfidtgfsl pfykrmlskk ltikdlesid tefynsliwi rdnnieecgl |
| 721 | emyfsvdmei lgkvtshdlk lggsnilvte enkdeyiglm tewrfsrgvq eqtkafldgf |
| 781 | nevvplqwlq yfdekelevm lcgmgevdla dwqrntvyrh ytrnskqiiw fwqfvketdn |
| 841 | evrmrllqfv tgtcrlplgg faelmgsngp qkfciekvgk dtwlprshtc fnrldlppyk |
| 901 | syeglkekll faieetegfg qe |

By “WWP1 polynucleotide” is meant a nucleic acid molecule encoding a WWP1 polypeptide. An exemplary WWP1 polynucleotide sequence is provided at NCBI Reference Sequence: NM_007013, and reproduced herein below (SEQ ID NO: 6):

| | |
|---|---|
| 1 | ggctgctggc ggcctgggct gccggggccg acgcctgggt ggctgctgcc gccgcgcctg |
| 61 | ctgcgagatg gcgatcttgg gcgcggaagg gtgagggcgc ccgccgcagg aggaggtgcc |
| 121 | gctgccgtgg ccgcccggct gccgggagcc gacagcttcg cgccggggtt gtctcctcac |
| 181 | agactatgag ctccttgaaa gagggaatcg tgtcttactc atctttgtat ccccagtgtc |
| 241 | tagcagttcc tgatacatag ttttagctga attttgggac atggccactg cttcaccaag |
| 301 | gtctgatact agtaataacc acagtggaag gttgcagtta caggtaactg tttctagtgc |
| 361 | caaacttaaa agaaaaaaga actggttcgg aacagcaata tatacagaag tagttgtaga |
| 421 | tggagaaatt acgaaaacag caaaatccag tagttcttct aatccaaaat gggatgaaca |
| 481 | gctaactgta aatgttacgc cacagactac attggaattt caagtttgga gccatcgcac |
| 541 | tttaaaagca gatgctttat taggaaaagc aacgatagat ttgaaacaag ctctgttgat |
| 601 | acacaataga aaattggaaa gagtgaaaga acaattaaaa ctttccttgg aaaacaagaa |
| 661 | tggcatagca caaactggtg aattgacagt tgtgcttgat ggattggtga ttgagcaaga |
| 721 | aaatataaca aactgcagct catctccaac catagaaata caggaaaatg gtgatgcctt |
| 781 | acatgaaaat ggagagcctt cagcaaggac aactgccagg ttggctgttg aaggcacgaa |
| 841 | tggaatagat aatcatgtac ctacaagcac tctagtccaa aactcatgct gctcgtatgt |
| 901 | agttaatgga gacaacacac cttcatctcc gtctcaggtt gctgccagac ccaaaaatac |
| 961 | accagctcca aaaccactcg catctgagcc tgccgatgac actgttaatg gagaatcatc |
| 1021 | ctcatttgca ccaactgata atgcgtctgt cacgggtact ccagtagtgt ctgaagaaaa |
| 1081 | tgccttgtct ccaaattgca ctagtactac tgttgaagat cctccagttc aagaaatact |
| 1141 | gacttcctca gaaaacaatg aatgtattcc ttctaccagt gcagaattgg aatctgaagc |
| 1201 | tagaagtata ttagagcctg acacctctaa ttctagaagt agttctgctt ttgaagcagc |
| 1261 | caaatcaaga cagccagatg ggtgtatgga tcctgtacgg cagcagtctg ggaatgccaa |
| 1321 | cacagaaacc ttgccatcag ggtgggaaca aagaaaagat cctcatggta gaacctatta |
| 1381 | tgtggatcat aatactcgaa ctaccacatg ggagagacca caacctttac ctccaggttg |
| 1441 | ggaaagaaga gttgatgatc gtagaagagt ttattatgtg gatcataaca ccagaacaac |
| 1501 | aacgtggcag cggcctacca tggaatctgt ccgaaatttt gaacagtggc aatctcagcg |
| 1561 | gaaccaattg cagggagcta tgcaacagtt taaccaacga tacctctatt cggcttcaat |
| 1621 | gttagctgca gaaaatgacc cttatggacc tttgccacca ggctgggaaa aaagagtgga |
| 1681 | ttcaacagac agggtttact ttgtgaatca taacacaaaa acaacccagt gggaagatcc |
| 1741 | aagaactcaa ggcttacaga atgaagaacc cctgccagaa ggctgggaaa ttagatatac |
| 1801 | tcgtgaaggt gtaaggtact ttgttgatca taacacaaga acaacaacat tcaaagatcc |
| 1861 | tcgcaatggg aagtcatctg taactaaagg tggtccacaa attgcttatg aacgcggctt |
| 1921 | taggtggaag cttgctcact tccgttattt gtgccagtct aatgcactac ctagtcatgt |
| 1981 | aaagatcaat gtgtcccggc agacattgtt tgaagattcc ttccaacaga ttatggcatt |
| 2041 | aaaaccctat gacttgagga ggcgcttata tgtaatattt agaggagaag aaggacttga |
| 2101 | ttatggtggc ctagcgagag aatggttttt cttgctttca catgaagttt tgaacccaat |
| 2161 | gtattgctta tttgagtatg cgggcaagaa caactattgt ctgcagataa atccagcatc |
| 2221 | aaccattaat ccagaccatc tttcatactt ctgtttcatt ggtcgtttta ttgccatggc |
| 2281 | actatttcat ggaaagttta tcgatactgg tttctcttta ccattctaca agcgtatgtt |
| 2341 | aagtaaaaaa cttactatta aggatttgga atctattgat actgaatttt ataactccct |
| 2401 | tatctggata agagataaca acattgaaga atgtggctta gaaatgtact tttctgttga |
| 2461 | catggagatt ttgggaaaag ttacttcaca tgacctgaag ttgggaggtt ccaatattct |
| 2521 | ggtgactgag gagaacaaag atgaatatat tggtttaatg acagaatggc gtttttctcg |
| 2581 | aggagtacaa gaacagacca aagctttcct tgatggtttt aatgaagttg ttcctcttca |
| 2641 | gtggctacag tacttcgatg aaaaagaatt agaggttatg ttgtgtggca tgcaggaggt |
| 2701 | tgacttggca gattggcaga gaaatactgt ttatcgacat tatacaagaa acagcaagca |
| 2761 | aatcatttgg ttttggcagt ttgtgaaaga gacagacaat gaagtaagaa tgcgactatt |
| 2821 | gcagttcgtc actggaacct gccgtttacc tctaggagga tttgctgagc tcatgggaag |
| 2881 | taatgggcct caaaagtttt gcattgaaaa agttggcaaa gacacttggt taccaagaag |
| 2941 | ccatacatgt tttaatcgct tggatctacc accatataag agttatgaac aactaaagga |
| 3001 | aaaacttctt tttgcaatag aagagacaga gggatttgga caagaatgaa tgtggcttct |
| 3061 | tattttggag gagctcttgc atttaaatac cccagccaag aaaaattgca cagatagtgt |
| 3121 | atataagctg ttcattctgt acagtgaatt ttccgaacct ctcaaagtat gttttccgtt |
| 3181 | cttccacaga aatatgcaaa acagttcatc cttttctact ttatttattg ttcccttgaa |
| 3241 | atgactgacc aggaaaaaga tcatccttaa attttgaagc aagtgagaga ctttattaaa |
| 3301 | aatacatata tatctatata aacatatatg atagtggctc tagttttata gagctccaag |
| 3361 | tgtattaaac atgacagcca ttcattcata aagatctgga tttgctttac cttgttaata |
| 3421 | ttatctaggg gaaaaagtgc aaattgctcc atgttcttct ctcccttatg taacatctcc |
| 3481 | tgagggtgtt tagttgcatg gctgttcaga aaggtattaa gggcttaggc caaatcttac |
| 3541 | tttgagtatg ttaaaaaaaa aaaaatgctg ctggcttttc tgaagacagg tgcttgaact |
| 3601 | tgtcagtttg ttttaaataa atacaatagt tgaaaatttt tctctgttac atcagtaata |
| 3661 | ttgttaaagt aatggataga accataactt acacatgaaa gtcatatact agatccaata |
| 3721 | ctatttagtt tattatcgaa attggaagga ttcattgagc agcatagaag tttgtttaca |
| 3781 | tgttactttg agatgctagg tatttgtgga attaaaaaga atcaggctct tttgtacttt |
| 3841 | gtttttaaat ctgtgatgct tttcaaattt aattcataat aaattgatgc aatttcatac |
| 3901 | ttaggaacat acaaaaggta atgtaaactc tgccactttt ttgtgttcaa aattttggtt |
| 3961 | tttatgaagc cagatggatt gaagagttac ataagcattt gaatgctcta atataaggct |
| 4021 | aatgattttc tgttagtgtt tgaatatctt cattcctctc aaattcataa cagttctatt |
| 4081 | taactgaatt aaataaccat atgaaaaaaa aaaaaaaaaa |

By “agent” is meant any small molecule chemical compound, antibody, nucleic acid molecule, or polypeptide, or fragments thereof.

By “ameliorate” is meant decrease, suppress, attenuate, diminish, arrest, or stabilize the development or progression of a disease.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of the analyte to be detected.

By “disease” is meant any condition or disorder that damages or interferes with the normal function of a cell, tissue, or organ. Examples of diseases include any cancer, including but not limited to breast cancer, prostate cancer, and colon cancer.

By “effective amount” is meant the amount of a required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

The invention provides a number of targets that are useful for the development of highly specific drugs to treat or a disorder characterized by the methods delineated herein. In addition, the methods of the invention provide a facile means to identify therapies that are safe for use in subjects. In addition, the methods of the invention provide a route for analyzing virtually any number of compounds for effects on a disease described herein with high-volume throughput, high sensitivity, and low complexity.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “inhibitory nucleic acid” is meant a double-stranded RNA, siRNA, shRNA, or antisense RNA, or a portion thereof, or a mimetic thereof, that when administered to a mammalian cell results in a decrease (e.g., by 10%, 25%, 50%, 75%, or even 90-100%) in the expression of a target gene. Typically, a nucleic acid inhibitor comprises at least a portion of a target nucleic acid molecule, or an ortholog thereof, or comprises at least a portion of the complementary strand of a target nucleic acid molecule. For example, an inhibitory nucleic acid molecule comprises at least a portion of any or all of the nucleic acids delineated herein.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any protein or polynucleotide having an alteration in expression level or activity that is associated with a disease or disorder. Cancers of the invention are those characterized by a reduction in, or an alteration in, or the loss of markers Pten and p53.

As used herein, “obtaining” as in “obtaining an agent” includes synthesizing, purchasing, or otherwise acquiring the agent.

By “reduces” is meant a negative alteration of at least 10%, 25%, 50%, 75%, or 100%.

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “siRNA” is meant a double stranded RNA. Optimally, an siRNA is 18, 19, 20, 21, 22, 23 or 24 nucleotides in length and has a 2 base overhang at its 3′ end. These dsRNAs can be introduced to an individual cell or to a whole animal; for example, they may be introduced systemically via the bloodstream. Such siRNAs are used to downregulate mRNA levels or promoter activity.

By “specifically binds” is meant a compound or antibody that recognizes and binds a polypeptide of the invention, but which does not substantially recognize and bind other molecules in a sample, for example, a biological sample, which naturally includes a polypeptide of the invention.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant a mammal, including, but not limited to, a human or non-human mammal, such as a bovine, equine, canine, ovine, or feline.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” “treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated. As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1 comprises gel images showing that WWP1 specifically interacts with PD-L1.

FIG. 2 comprises gel images showing that WWP1 triggers PD-L1 ubiquitination in a catalytic dependent manner.

FIG. 3 comprises gel images showing that WWP1 triggers PD-L1 K63 ubiquitination.

FIG. 4 is a gel image showing a positive correlation of WWP1 and PD-L1 in triple negative breast cancer.

FIG. 5 is a series of gel images and graphs that demonstrate that depletion of WWP1 robustly reduces PD-L1 expression.

FIGS. 6A to 6D illustrate the depletion of WWP1 dramatically decreases PD-L1 protein abundance in multiple human and mouse cancer cell lines. FIG. 6A is a gel image showing that depletion of WWP1 decreases PD-L1 protein in DU145 cells. FIG. 6B is a gel image showing that depletion of WWP1 decreases PD-L1 protein in HCT116 cells. FIG. 6C is a gel image showing that depletion of WWP1 decreases PD-L1 protein in DLD1 cells. FIG. 6D is a gel image showing that depletion of WWP1 decreases PD-L1 protein in CT26 cells.

FIG. 7 is a gel image showing that knockout of WWP1 robustly reduces PD-L1 expression.

FIGS. 8A and 8B illustrate that the depletion of WWP1 shortens PD-L1 half-life in both human and mouse cancer cell lines. FIG. 8A is a gel image showing the depletion of WWP1 shortens PD-L1 half-life in human HCT116 cells. FIG. 8B is a gel image showing the depletion of WWP1 shortens PD-L1 half-life in murine CT26 cells.

FIG. 9 is a gel image showing that depletion of WWP1 reduces PD-L1 protein stability.

FIG. 10 illustrates that WWP1 stabilizes PD-L1 in a catalytic dependent manner.

FIG. 11 is a series of gel images showing that WWP1 stabilizes PD-L1 in a catalytic dependent manner.

FIG. 12 is a gel images that demonstrates that MG132 treatment restores PD-L1 protein levels in WWP1 KO cells.

FIG. 13 illustrates that WWP1-mediated PD-L1 downregulation is predominately through proteasome, but not through autophagy.

FIG. 14 is a gel image showing that WWP1-mediated PD-L1 K63-Ub(n) competes for K48-Ub(n).

FIG. 15 illustrates that WWP1-mediated PD-L1 degradation induces active CD8 T cell infiltration into tumors (Syngenic mice model).

FIG. 16 illustrates that WWP1 inhibition by I3C triggers active CD8 T cell tumor infiltration (Syngenic mice model).

FIG. 17 shows the results of a gene ontology (GO) analysis of the intersect of downregulated genes in comparisons of knockout (KO) versus wildtype (WT) and treatment (TX) versus vehicle (VEH).

FIG. 18 are charts (knockout versus wildtype and IC3 versus vehicle) illustrating that the JAX-STAT pathway is down regulated with WWP1 inhibition.

FIG. 19 are charts (knockout versus wildtype and IC3 versus vehicle) illustrating that the NF-kB pathway is down regulated with WWP1 inhibition.

FIG. 20 are charts (knockout versus wildtype and IC3 versus vehicle) illustrating that the TNFα pathway is down regulated with WWP1 inhibition.

FIG. 21 is a graph illustrating that genetic or pharmacological inactivation of WWP1 decreases PD-L2 mRNA levels, but not PD-L1 mRNA.

FIG. 22 is an illustration depicting enhancing an anti-tumor immune response by inhibiting levels or activity of WWP1 and/or PD-L1.

FIG. 23 is an illustration showing that WWP1 is a druggable double-edge cancer promoting sword.

### DETAILED DESCRIPTION OF THE INVENTION

The invention features compositions and methods that are useful for the treatment of cancer (e.g., breast cancer, colon adenocarcinoma, prostate cancer) using an agent that inhibits the expression or activity of WW domain-containing protein-1 (WWP1) alone or in combination with PD1/PD-L1 blockade.

The invention is based, at least in part, on the discovery that levels of WWP1 and PD-L1 correlate, and that WWP1 mediates PD-L1 degradation via the proteasome. This degradation as well as inhibition of WWP1 by indol 3 carbinol (I3C) induced active CD8 T cell infiltration into tumors, thereby reducing tumor size.

### Inhibitory Nucleic Acids

Inhibitory nucleic acid molecules are those oligonucleotides that inhibit the expression or activity of NEDD4-1 or WWP1. These inhibitory oligonucleotides are useful in combination with PD-1/PD-L1 blockade (e.g., anti-PD1, anti-PDL1 antibodies) for the treatment of cancer. Such oligonucleotides include single and double stranded nucleic acid molecules (e.g., DNA, RNA, and analogs thereof) that bind a nucleic acid molecule that encodes a NEDD4-1 or WWP1 polypeptide (e.g., antisense molecules, siRNA, shRNA), as well as nucleic acid molecules that bind directly to the polypeptide to modulate its biological activity (e.g., aptamers). Inhibitory nucleic acid molecules described herein are useful for the treatment of cancer (e.g., (e.g., bladder cancer, breast cancer, colon adenocarcinoma, gastric adenocarcinoma, prostate cancer, liver cancer).

siRNA

Short twenty-one to twenty-five nucleotide double-stranded RNAs are effective at down-regulating gene expression (Zamore et al., Cell 101: 25-33; Elbashir et al., Nature 411: 494-498, 2001, hereby incorporated by reference). The therapeutic effectiveness of an sirNA approach in mammals was demonstrated in vivo by McCaffrey et al. (Nature 418: 38-39.2002).

Given the sequence of a target gene, siRNAs may be designed to inactivate that gene. Such siRNAs, for example, could be administered directly to an affected tissue, or administered systemically. The nucleic acid sequence of a gene can be used to design small interfering RNAs (siRNAs). The 21 to 25 nucleotide siRNAs may be used, for example, as therapeutics to treat cancer.

The inhibitory nucleic acid molecules of the present invention may be employed as double-stranded RNAs for RNA interference (RNAi)-mediated knock-down of expression. In one embodiment, expression of NEDD4-1 polypeptide and/or WWP1 polypeptide is reduced in a subject having cancer. RNAi is a method for decreasing the cellular expression of specific proteins of interest (reviewed in Tuschl, Chembiochem 2:239-245, 2001; Sharp, Genes & Devel. 15:485-490, 2000; Hutvagner and Zamore, Curr. Opin. Genet. Devel. 12:225-232, 2002; and Hannon, Nature 418:244-251, 2002). The introduction of siRNAs into cells either by transfection of dsRNAs or through expression of siRNAs using a plasmid-based expression system is increasingly being used to create loss-of-function phenotypes in mammalian cells.

In one embodiment of the invention, a double-stranded RNA (dsRNA) molecule is made that includes between eight and nineteen consecutive nucleobases of a nucleobase oligomer of the invention. The dsRNA can be two distinct strands of RNA that have duplexed, or a single RNA strand that has self-duplexed (small hairpin (sh)RNA). Typically, dsRNAs are about 21 or 22 base pairs, but may be shorter or longer (up to about 29 nucleobases) if desired. dsRNA can be made using standard techniques (e.g., chemical synthesis or in vitro transcription). Kits are available, for example, from Ambion (Austin, Tex.) and Epicentre (Madison, Wis.). Methods for expressing dsRNA in mammalian cells are described in Brummelkamp et al. Science 296:550-553, 2002; Paddison et al. Genes & Devel. 16:948-958, 2002. Paul et al. Nature Biotechnol. 20:505-508, 2002; Sui et al. Proc. Natl. Acad. Sci. USA 99:5515-5520, 2002; Yu et al. Proc. Natl. Acad. Sci. USA 99:6047-6052, 2002; Miyagishi et al. Nature Biotechnol. 20:497-500, 2002; and Lee et al. Nature Biotechnol. 20:500-505 2002, each of which is hereby incorporated by reference.

Small hairpin RNAs (shRNAs) comprise an RNA sequence having a stem-loop structure. A “stem-loop structure” refers to a nucleic acid having a secondary structure that includes a region of nucleotides which are known or predicted to form a double strand or duplex (stem portion) that is linked on one side by a region of predominantly single-stranded nucleotides (loop portion). The term “hairpin” is also used herein to refer to stem-loop structures. Such structures are well known in the art and the term is used consistently with its known meaning in the art. As is known in the art, the secondary structure does not require exact base-pairing. Thus, the stem can include one or more base mismatches or bulges. Alternatively, the base-pairing can be exact, i.e. not include any mismatches. The multiple stem-loop structures can be linked to one another through a linker, such as, for example, a nucleic acid linker, a miRNA flanking sequence, other molecule, or some combination thereof.

As used herein, the term “small hairpin RNA” includes a conventional stem-loop shRNA, which forms a precursor miRNA (pre-miRNA). While there may be some variation in range, a conventional stem-loop shRNA can comprise a stem ranging from 19 to 29 bp, and a loop ranging from 4 to 30 bp. “shRNA” also includes micro-RNA embedded shRNAs (miRNA-based shRNAs), wherein the guide strand and the passenger strand of the miRNA duplex are incorporated into an existing (or natural) miRNA or into a modified or synthetic (designed) miRNA. In some instances the precursor miRNA molecule can include more than one stem-loop structure. MicroRNAs are endogenously encoded RNA molecules that are about 22-nucleotides long and generally expressed in a highly tissue- or developmental-stage-specific fashion and that post-transcriptionally regulate target genes. More than 200 distinct miRNAs have been identified in plants and animals. These small regulatory RNAs are believed to serve important biological functions by two prevailing modes of action: (1) by repressing the translation of target mRNAs, and (2) through RNA interference (RNAi), that is, cleavage and degradation of mRNAs. In the latter case, miRNAs function analogously to small interfering RNAs (siRNAs). Thus, one can design and express artificial miRNAs based on the features of existing miRNA genes.

shRNAs can be expressed from DNA vectors to provide sustained silencing and high yield delivery into almost any cell type. In some embodiments, the vector is a viral vector. Exemplary viral vectors include retroviral, including lentiviral, adenoviral, baculoviral and avian viral vectors, and including such vectors allowing for stable, single-copy genomic integrations. Retroviruses from which the retroviral plasmid vectors can be derived include, but are not limited to, Moloney Murine Leukemia Virus, spleen necrosis virus, Rous sarcoma Virus, Harvey Sarcoma Virus, avian leukosis virus, gibbon ape leukemia virus, human immunodeficiency virus, Myeloproliferative Sarcoma Virus, and mammary tumor virus. A retroviral plasmid vector can be employed to transduce packaging cell lines to form producer cell lines. Examples of packaging cells which can be transfected include, but are not limited to, the PE501, PA317, R-2, R-AM, PA12, T19-14x, VT-19-17-H2, RCRE, RCRIP, GP+E-86, GP+envAm12, and DAN cell lines as described in Miller, Human Gene Therapy 1:5-14 (1990), which is incorporated herein by reference in its entirety. The vector can transduce the packaging cells through any means known in the art. A producer cell line generates infectious retroviral vector particles which include polynucleotide encoding a DNA replication protein. Such retroviral vector particles then can be employed, to transduce eukaryotic cells, either in vitro or in vivo. The transduced eukaryotic cells will express a DNA replication protein.

Catalytic RNA molecules or ribozymes that include an antisense sequence of the present invention can be used to inhibit expression of a nucleic acid molecule in vivo (e.g., a nucleic acid encoding NEDD4-1 or WWP1). The inclusion of ribozyme sequences within antisense RNAs confers RNA-cleaving activity upon them, thereby increasing the activity of the constructs. The design and use of target RNA-specific ribozymes is described in Haseloff et al., Nature 334:585-591. 1988, and U.S. Patent Application Publication No. 2003/0003469 A1, each of which is incorporated by reference.

Accordingly, the invention also features a catalytic RNA molecule that includes, in the binding arm, an antisense RNA having between eight and nineteen consecutive nucleobases. In preferred embodiments of this invention, the catalytic nucleic acid molecule is formed in a hammerhead or hairpin motif. Examples of such hammerhead motifs are described by Rossi et al., Aids Research and Human Retroviruses, 8:183, 1992. Example of hairpin motifs are described by Hampel et al., “RNA Catalyst for Cleaving Specific RNA Sequences,” filed Sep. 20, 1989, which is a continuation-in-part of U.S. Ser. No. 07/247,100 filed Sep. 20, 1988, Hampel and Tritz, Biochemistry, 28:4929, 1989, and Hampel et al., Nucleic Acids Research, 18: 299, 1990. These specific motifs are not limiting in the invention and those skilled in the art will recognize that all that is important in an enzymatic nucleic acid molecule of this invention is that it has a specific substrate binding site which is complementary to one or more of the target gene RNA regions, and that it have nucleotide sequences within or surrounding that substrate binding site which impart an RNA cleaving activity to the molecule.

Alternatively, expression of NEDD4-1, WWP1, or both, may be inhibited, or silenced by introducing vectors encoding Clustered regularly interspaced short palindromic repeats (CRISPR)/Cas9 nuclease engineered to target NEDD4-1, WWP1, or both.

Essentially any method for introducing a nucleic acid construct into cells can be employed. Physical methods of introducing nucleic acids include injection of a solution containing the construct, bombardment by particles covered by the construct, soaking a cell, tissue sample or organism in a solution of the nucleic acid, or electroporation of cell membranes in the presence of the construct. A viral construct packaged into a viral particle can be used to accomplish both efficient introduction of an expression construct into the cell and transcription of the encoded shRNA. Other methods known in the art for introducing nucleic acids to cells can be used, such as lipid-mediated carrier transport, chemical mediated transport, such as calcium phosphate, and the like. Thus the shRNA-encoding nucleic acid construct can be introduced along with components that perform one or more of the following activities: enhance RNA uptake by the cell, promote annealing of the duplex strands, stabilize the annealed strands, or otherwise increase inhibition of the target gene.

For expression within cells, DNA vectors, for example plasmid vectors comprising either an RNA polymerase II or RNA polymerase III promoter can be employed. Expression of endogenous miRNAs is controlled by RNA polymerase II (Pol II) promoters and in some cases, shRNAs are most efficiently driven by Pol II promoters, as compared to RNA polymerase III promoters (Dickins et al., 2005, Nat. Genet. 39: 914-921). In some embodiments, expression of the shRNA can be controlled by an inducible promoter or a conditional expression system, including, without limitation, RNA polymerase type II promoters. Examples of useful promoters in the context of the invention are tetracycline-inducible promoters (including TRE-tight), IPTG-inducible promoters, tetracycline transactivator systems, and reverse tetracycline transactivator (rtTA) systems. Constitutive promoters can also be used, as can cell- or tissue-specific promoters. Many promoters will be ubiquitous, such that they are expressed in all cell and tissue types. A certain embodiment uses tetracycline-responsive promoters, one of the most effective conditional gene expression systems in in vitro and in vivo studies. See International Patent Application PCT/US2003/030901 (Publication No. WO 2004-029219 A2) and Fewell et al., 2006, Drug Discovery Today 11: 975-982, for a description of inducible shRNA.

### Delivery of Polynucleotides

Naked polynucleotides, or analogs thereof, are capable of entering mammalian cells and inhibiting expression of a gene of interest (e.g., a NEDD4-1 or WWP1 polynucleotide). Nonetheless, it may be desirable to utilize a formulation that aids in the delivery of oligonucleotides or other nucleobase oligomers to cells (see, e.g., U.S. Pat. Nos. 5,656,611, 5,753,613, 5,785,992, 6,120,798, 6,221,959, 6,346,613, and 6,353,055, each of which is hereby incorporated by reference).

### Small Molecule Inhibitors

The invention provides small molecules capable of inhibiting NEDD4-1 and/or WWP1 activity that are useful for the treatment of cancer. Such compounds may be used in combination with anti-PD1, anti-PDL1 antibodies. Examples of compounds suitable as a NEDD4-1 inhibitor include 4-(4-chlorobenzoyl) piperazin-1-yl) (4-(phonoxymethyl) phenyl) methanone.

Another example of a compound suitable as a NEDD4-1 inhibitor is indole-3-carbinol (I3C). The structure of I3C is shown below:

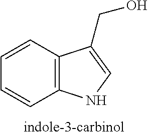 [SMILES: OCc1c[nH]c2ccccc12]

Another example of compounds suitable as NEDD4-1 inhibitors are the compounds listed in U.S. Patent Application No. US20140179637 A1 (incorporated by reference in its entirety).

### Therapeutic Methods

The methods and compositions provided herein can be used to treat or prevent progression of a cancer (e.g., bladder cancer, breast cancer, colon adenocarcinoma, gastric adenocarcinoma, prostate cancer, liver cancer). In general, an effective amount of at least one agent selected from the group consisting of: an agent that inhibits the expression or activity of neural precursor cell expressed developmentally down-regulated protein 4 (NEDD4-1), and an agent that inhibits the expression or activity of WW domain-containing protein-1 (WWP1) can be administered therapeutically and/or prophylactically.

Treatment will be suitably administered to subjects, particularly humans, suffering from, having, susceptible to, or at risk of developing such cancer. Determination of those subjects “at risk” can be made by any objective or subjective determination by a diagnostic test or opinion of a subject or health care provider (e.g., genetic test, enzyme or protein marker, family history, and the like). Identifying a subject in need of such treatment can be in the judgment of a subject or a health care professional and can be subjective (e.g. opinion) or objective (e.g. measurable by a test or diagnostic method).

In some aspects, the effective amount of at least one agent selected from the group consisting of: an agent that inhibits the expression or activity of neural precursor cell expressed developmentally down-regulated protein 4 (NEDD4-1), and an agent that inhibits the expression or activity of WW domain-containing protein-1 (WWP1) may be administered in combination with one or more of any other standard anti-cancer therapies, such as anti-PD1 and/or anti-PDL1 antibodies. For example, an agent as described herein may be administered in combination with standard chemotherapeutics. Methods for administering combination therapies (e.g., concurrently or otherwise) are known to the skilled artisan and are described for example in Remington's Pharmaceutical Sciences by E. W. Martin.

### Combination Therapy

The invention provides for the use of PD-1/PDL-1 blockade in combination with an agent that inhibits NEDD4-1 or WWP1 expression or activity.

The invention further provides for the addition of chemotherapeutic agents suitable for use in the methods of the present invention include, but are not limited to alkylating agents. Without intending to be limited to any particular theory, alkylating agents directly damage DNA to keep the cell from reproducing. Alkylating agents work in all phases of the cell cycle and are used to treat many different cancers, including leukemia, lymphoma, Hodgkin disease, multiple myeloma, and sarcoma, as well as cancers of the lung, breast, and ovary.

Alkylating agents are divided into different classes, including, but not limited to: (i) nitrogen mustards, such as, for example mechlorethamine (nitrogen mustard), chlorambucil, cyclophosphamide (Cytoxan®), ifosfamide, and melphalan; (ii) nitrosoureas, such as, for example, streptozocin, carmustine (BCNU), and lomustine; (iii) alkyl sulfonates, such as, for example, busulfan; (iv) riazines, such as, for example, dacarbazine (DTIC) and temozolomide (Temodar®); (v) ethylenimines, such as, for example, thiotepa and altretamine (hexamethylmelamine); and (v) platinum drugs, such as, for example, cisplatin, carboplatin, and oxalaplatin.

### Pharmaceutical Compositions

The present invention features compositions useful for treating cancer comprising WWP1 inhibitors and/or NEDD4-1 inhibitors in combination with anti-PD1 and/or anti-PDL1 antibodies. The methods include administering to a subject having a cancer, an effective amount of at least one agent selected from the group consisting of: an agent that inhibits the expression or activity of neural precursor cell expressed developmentally down-regulated protein 4 (NEDD4-1), and an agent that inhibits the expression or activity of WW domain-containing protein-1 (WWP1) in a physiologically acceptable carrier. In particular embodiments, the invention provides for the use of WWP1 inhibitors and/or NEDD4-1 inhibitors in combination with anti-PD1 and/or anti-PDL1 antibodies.

Typically, the carrier or excipient for the composition provided herein is a pharmaceutically acceptable carrier or excipient, such as sterile water, aqueous saline solution, aqueous buffered saline solutions, aqueous dextrose solutions, aqueous glycerol solutions, ethanol, or combinations thereof. The preparation of such solutions ensuring sterility, pH, isotonicity, and stability is effected according to protocols established in the art. Generally, a carrier or excipient is selected to minimize allergic and other undesirable effects, and to suit the particular route of administration, e.g., subcutaneous, intramuscular, intranasal, and the like.

The administration may be by any suitable means that results in a concentration of the therapeutic that, combined with other components, is effective in ameliorating, reducing, or stabilizing the disease symptoms in a subject. The composition may be administered systemically, for example, formulated in a pharmaceutically-acceptable buffer such as physiological saline. Preferable routes of administration include, for example, subcutaneous, intravenous, interperitoneally, intramuscular, intrathecal, or intradermal injections that provide continuous, sustained levels of the agent in the patient. The amount of the therapeutic agent to be administered varies depending upon the manner of administration, the age and body weight of the patient, and with the clinical symptoms of the cancer. Generally, amounts will be in the range of those used for other agents used in the treatment of cancer, although in certain instances lower amounts will be needed because of the increased specificity of the agent. A composition is administered at a dosage that ameliorates or decreases effects of the cancer as determined by a method known to one skilled in the art.

The therapeutic or prophylactic composition may be contained in any appropriate amount in any suitable carrier substance, and is generally present in an amount of 1-95% by weight of the total weight of the composition. The composition may be provided in a dosage form that is suitable for parenteral (e.g., subcutaneously, intravenously, intramuscularly, intrathecally, or intraperitoneally) administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Pharmaceutical compositions according to the invention may be formulated to release the active agent substantially immediately upon administration or at any predetermined time or time period after administration. The latter types of compositions are generally known as controlled release formulations, which include (i) formulations that create a substantially constant concentration of the drug within the body over an extended period of time; (ii) formulations that after a predetermined lag time create a substantially constant concentration of the drug within the body over an extended period of time; (iii) formulations that sustain action during a predetermined time period by maintaining a relatively, constant, effective level in the body with concomitant minimization of undesirable side effects associated with fluctuations in the plasma level of the active substance (sawtooth kinetic pattern); (iv) formulations that localize action by, e.g., spatial placement of a controlled release composition adjacent to or in contact with an organ, such as the heart; (v) formulations that allow for convenient dosing, such that doses are administered, for example, once every one or two weeks; and (vi) formulations that target a disease using carriers or chemical derivatives to deliver the therapeutic agent to a particular cell type. For some applications, controlled release formulations obviate the need for frequent dosing during the day to sustain the plasma level at a therapeutic level.

Any of a number of strategies can be pursued in order to obtain controlled release in which the rate of release outweighs the rate of metabolism of the agent in question. In one example, controlled release is obtained by appropriate selection of various formulation parameters and ingredients, including, e.g., various types of controlled release compositions and coatings. Thus, the therapeutic is formulated with appropriate excipients into a pharmaceutical composition that, upon administration, releases the therapeutic in a controlled manner. Examples include single or multiple unit tablet or capsule compositions, oil solutions, suspensions, emulsions, microcapsules, microspheres, molecular complexes, nanoparticles, patches, and liposomes.

The pharmaceutical composition may be administered parenterally by injection, infusion or implantation (subcutaneous, intravenous, intramuscular, intraperitoneal, intrathecal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Remington: The Science and Practice of Pharmacy, supra.

Compositions for parenteral use may be provided in unit dosage forms (e.g., in single-dose ampoules), or in vials containing several doses and in which a suitable preservative may be added (see below). The composition may be in the form of a solution, a suspension, an emulsion, an infusion device, or a delivery device for implantation, or it may be presented as a dry powder to be reconstituted with water or another suitable vehicle before use. Apart from the active agent that reduces or ameliorates a cardiac dysfunction or disease, the composition may include suitable parenterally acceptable carriers and/or excipients. The active therapeutic agent(s) (e.g., at least one agent selected from the group consisting of: an agent that inhibits the expression or activity of neural precursor cell expressed developmentally down-regulated protein 4 (NEDD4-1), and an agent that inhibits the expression or activity of WW domain-containing protein-1 (WWP1) described herein) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing, agents.

In some embodiments, the composition comprising the active therapeutic is formulated for intravenous delivery. As indicated above, the pharmaceutical compositions according to the invention may be in the form suitable for sterile injection. To prepare such a composition, the suitable therapeutic(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate). In cases where one of the agents is only sparingly or slightly soluble in water, a dissolution enhancing or solubilizing agent can be added, or the solvent may include 10-60% w/w of propylene glycol or the like.

### Kits

The invention provides kits for the treatment or prevention of cancer. In some embodiments, the kit includes a therapeutic or prophylactic composition containing at least one agent selected from the group consisting of: an agent that inhibits the expression or activity of neural precursor cell expressed developmentally down-regulated protein 4 (NEDD4-1), and/or an agent that inhibits the expression or activity of WW domain-containing protein-1 (WWP1) in combination with an anti-PD1/anti-PDL1 antibody in unit dosage form. In other embodiments, the kit includes at least one agent selected from the group consisting of: an agent that inhibits the expression or activity of neural precursor cell expressed developmentally down-regulated protein 4 (NEDD4-1), and an agent that inhibits the expression or activity of WW domain-containing protein-1 (WWP1) in unit dosage form in a sterile container. Such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments.

If desired a pharmaceutical composition of the invention is provided together with instructions for administering the pharmaceutical composition to a subject having or at risk of contracting or developing cancer. The instructions will generally include information about the use of the composition for the treatment or prevention of cancer. In other embodiments, the instructions include at least one of the following: description of the therapeutic/prophylactic agent; dosage schedule and administration for treatment or prevention of cancer or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1

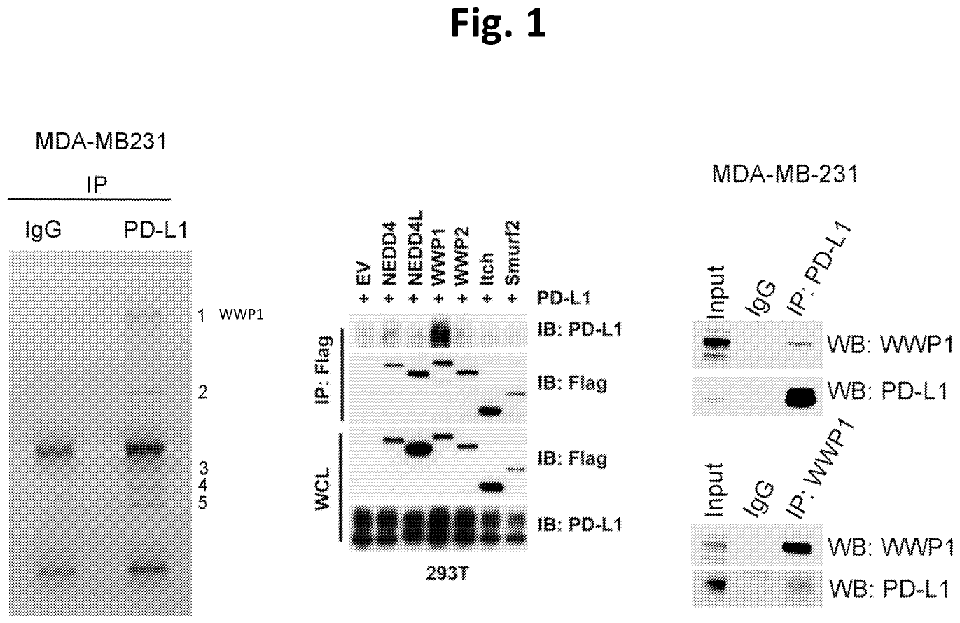

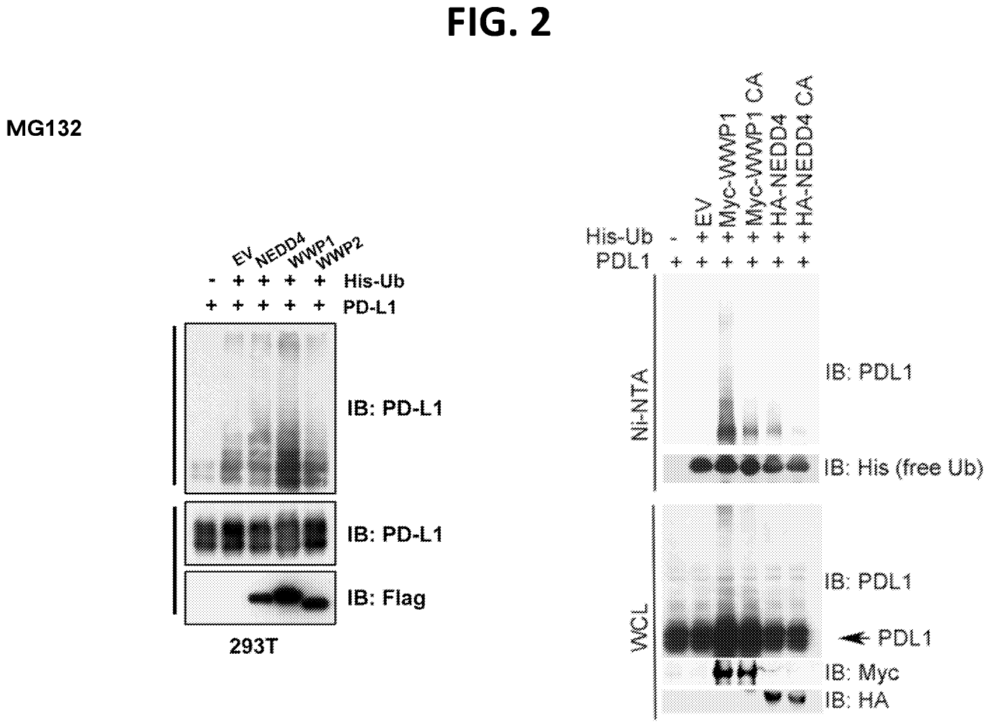

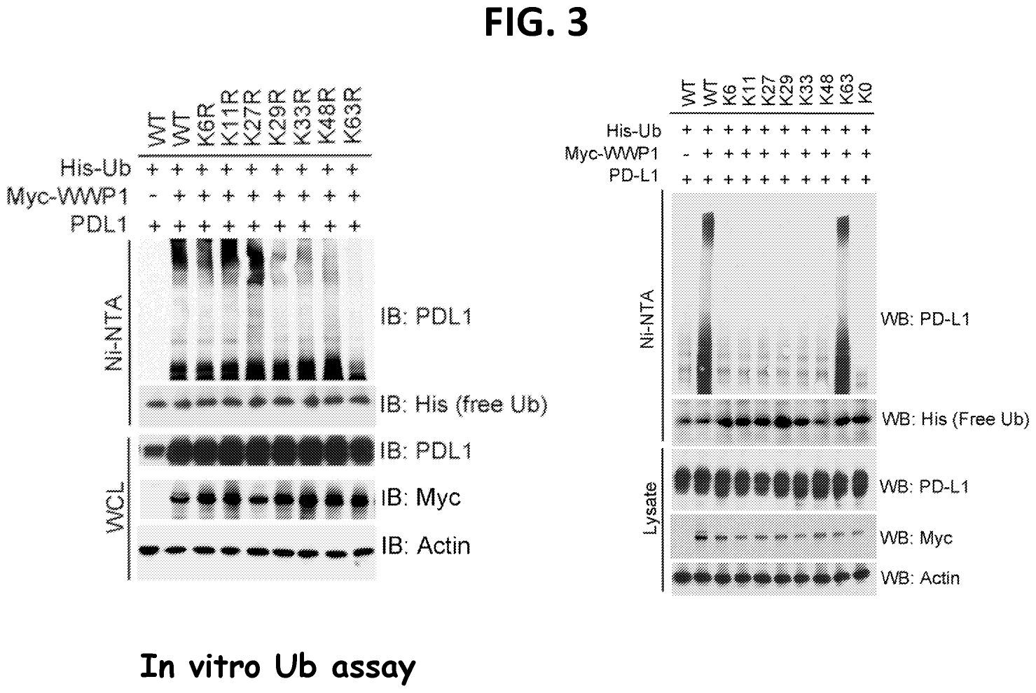

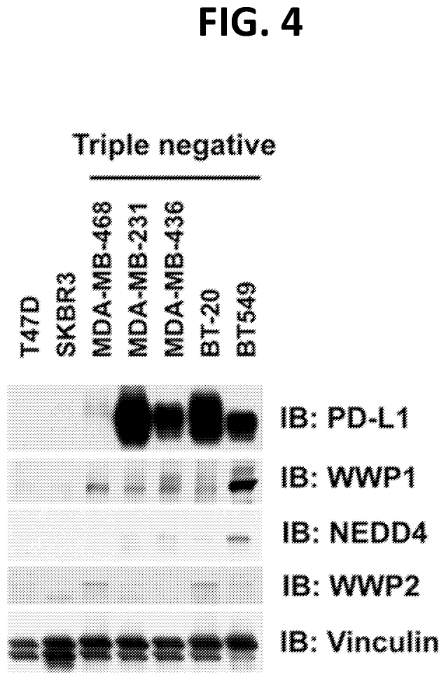

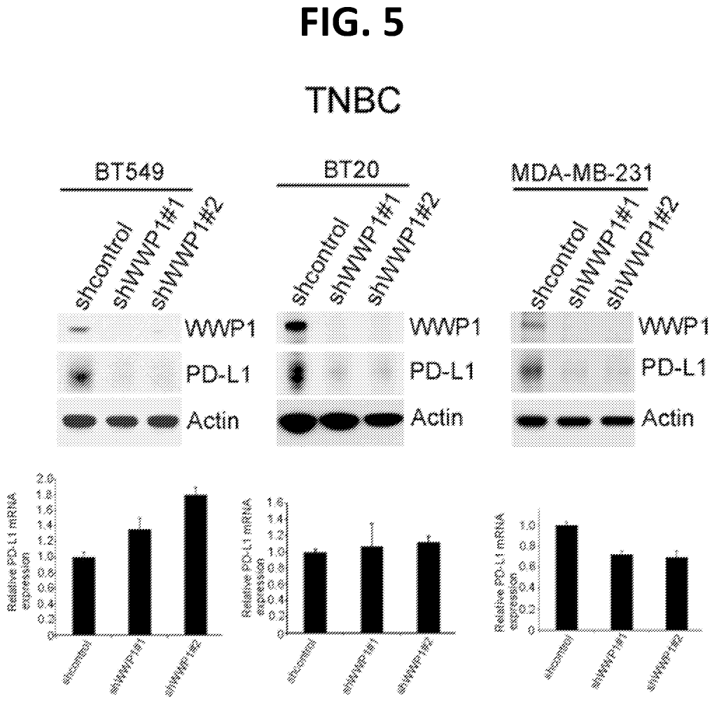

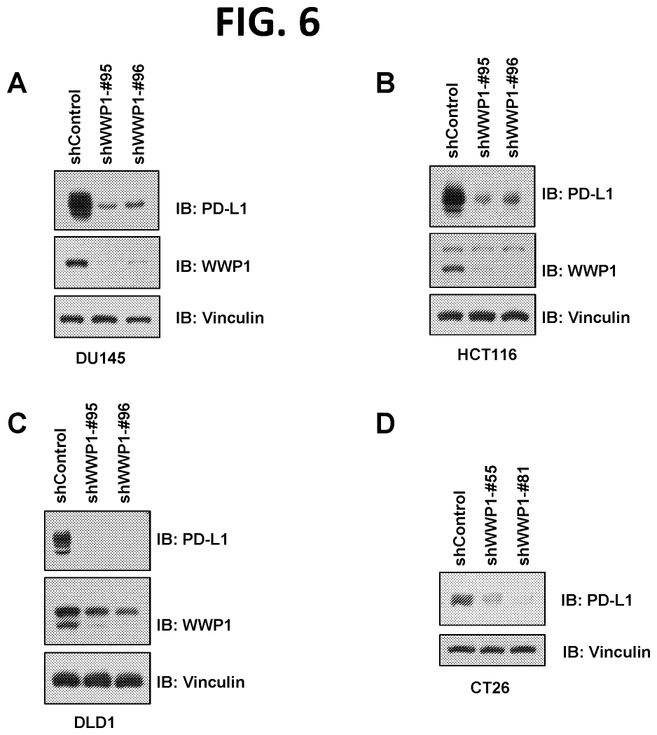

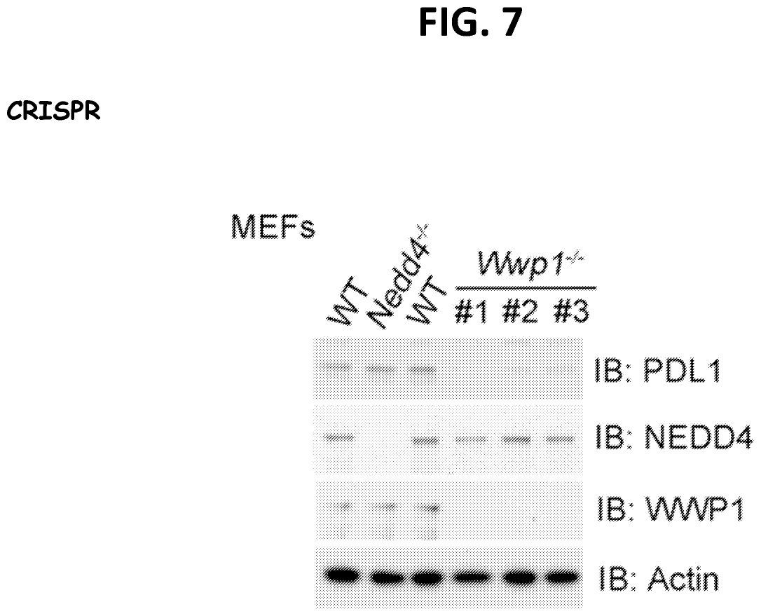

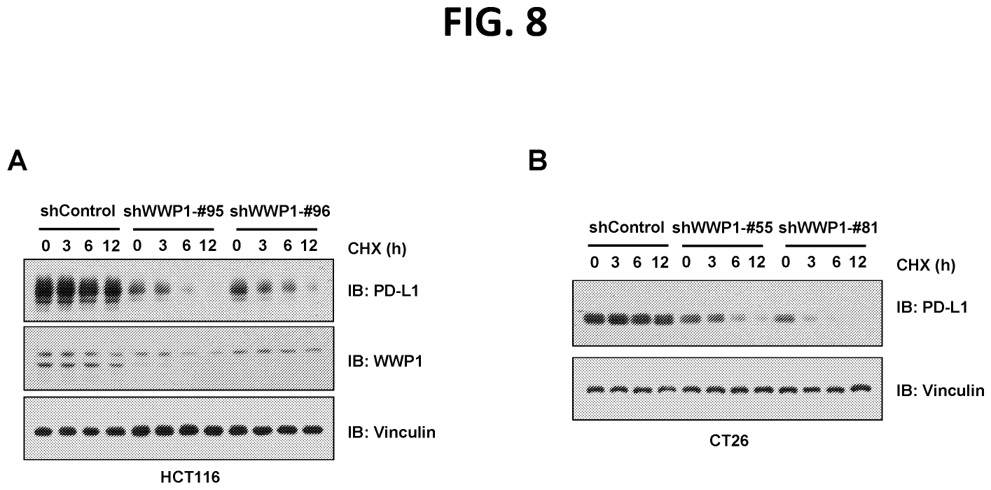

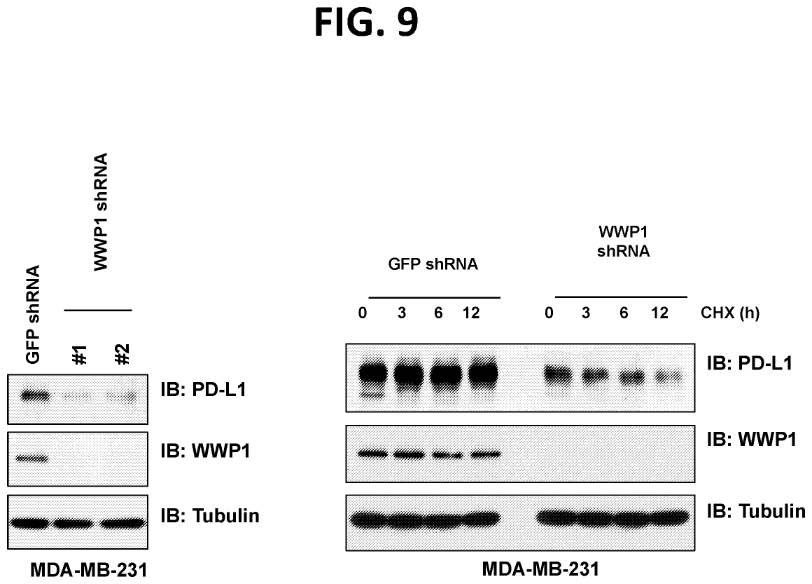

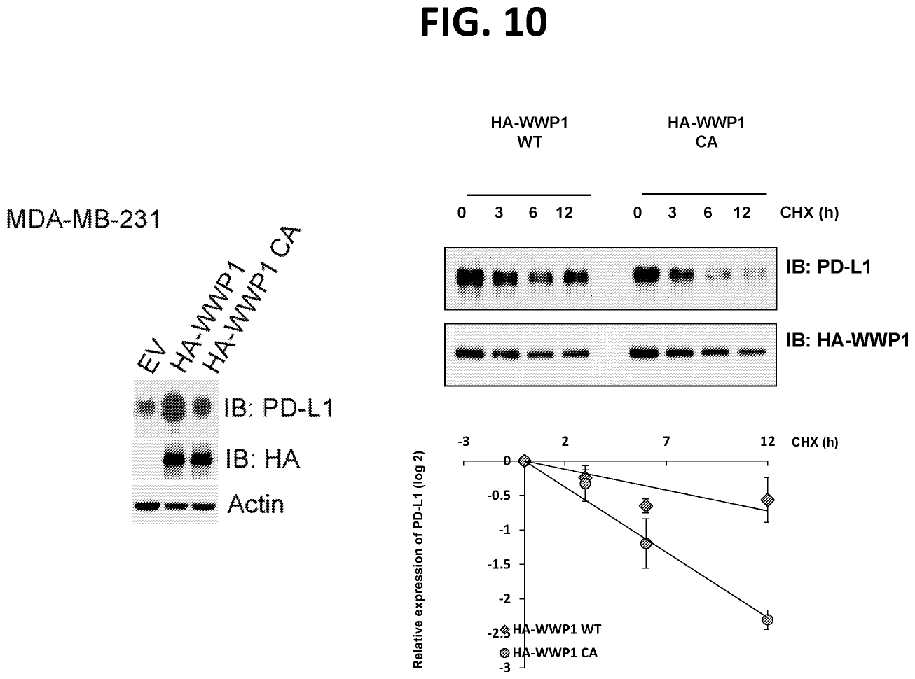

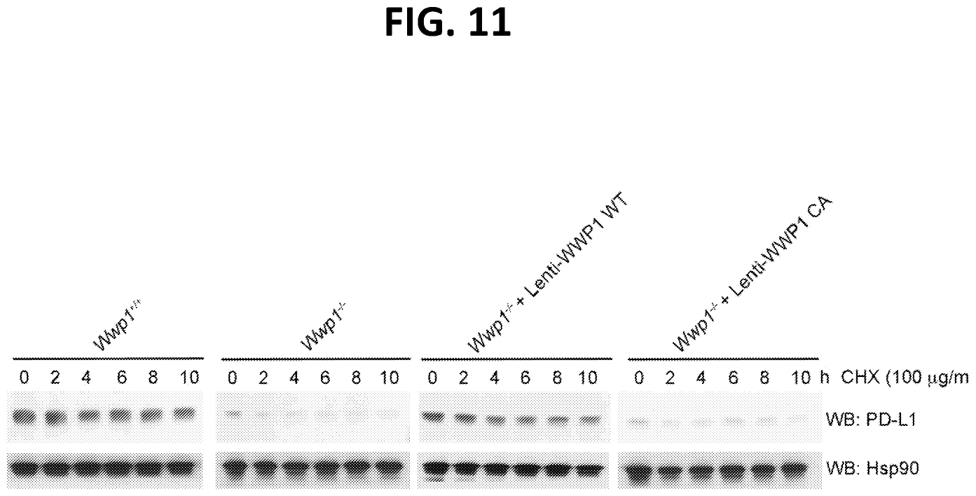

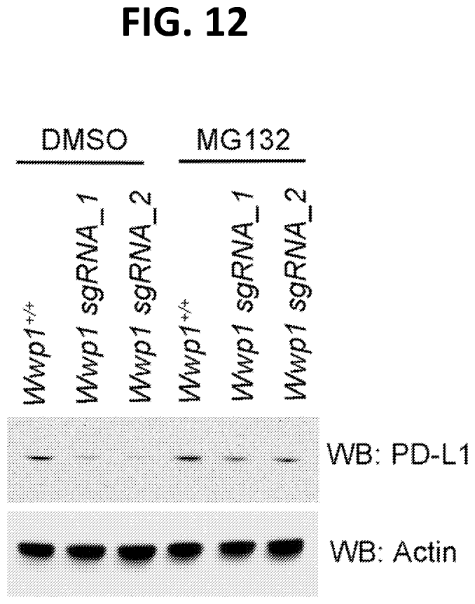

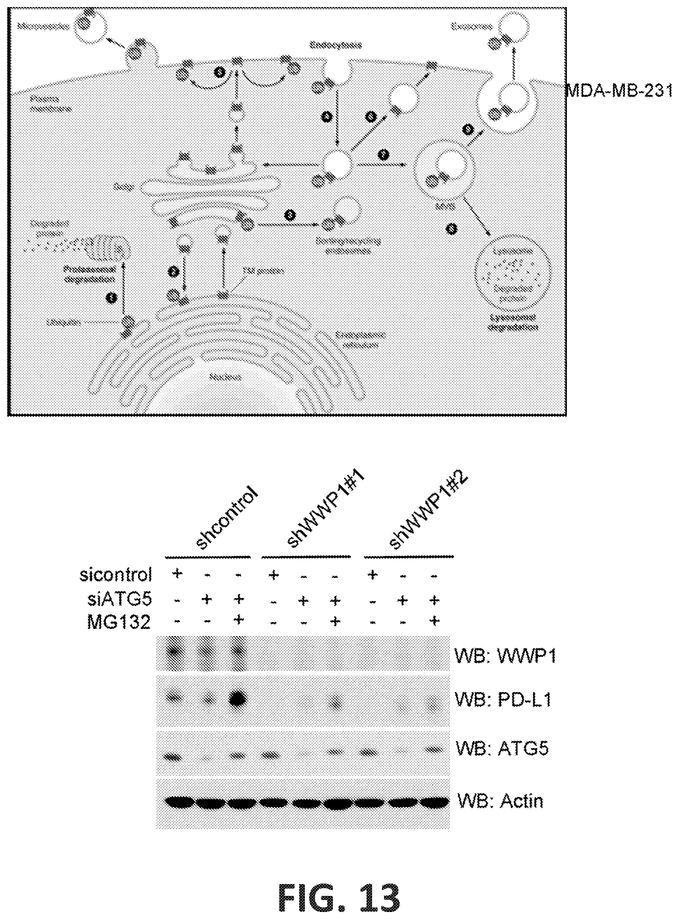

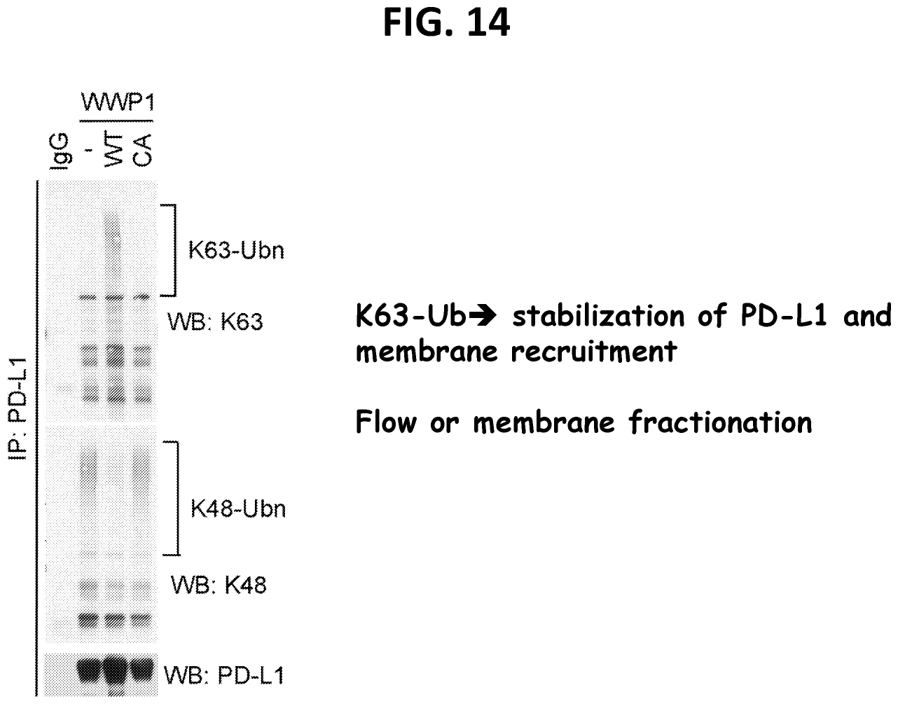

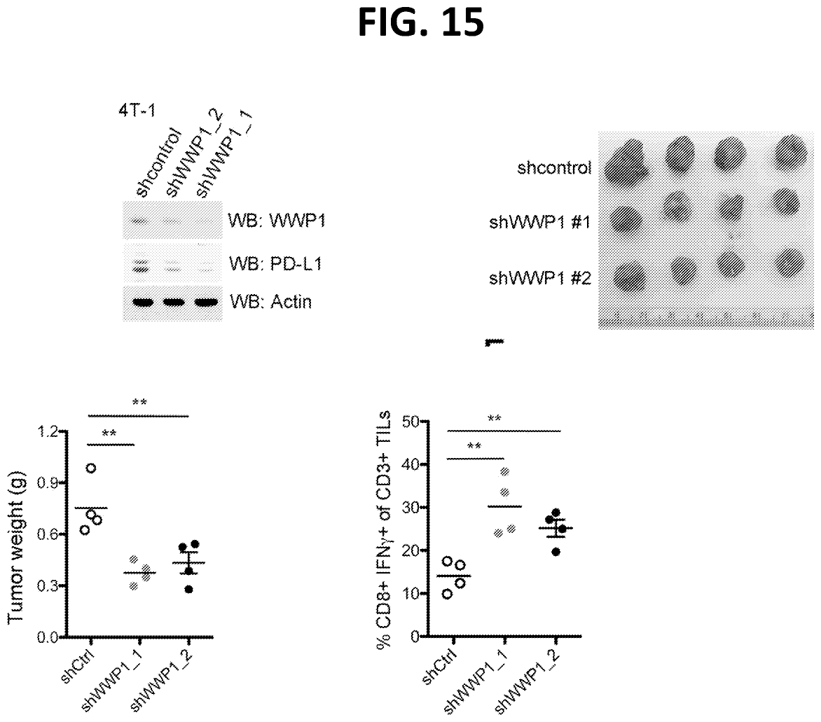

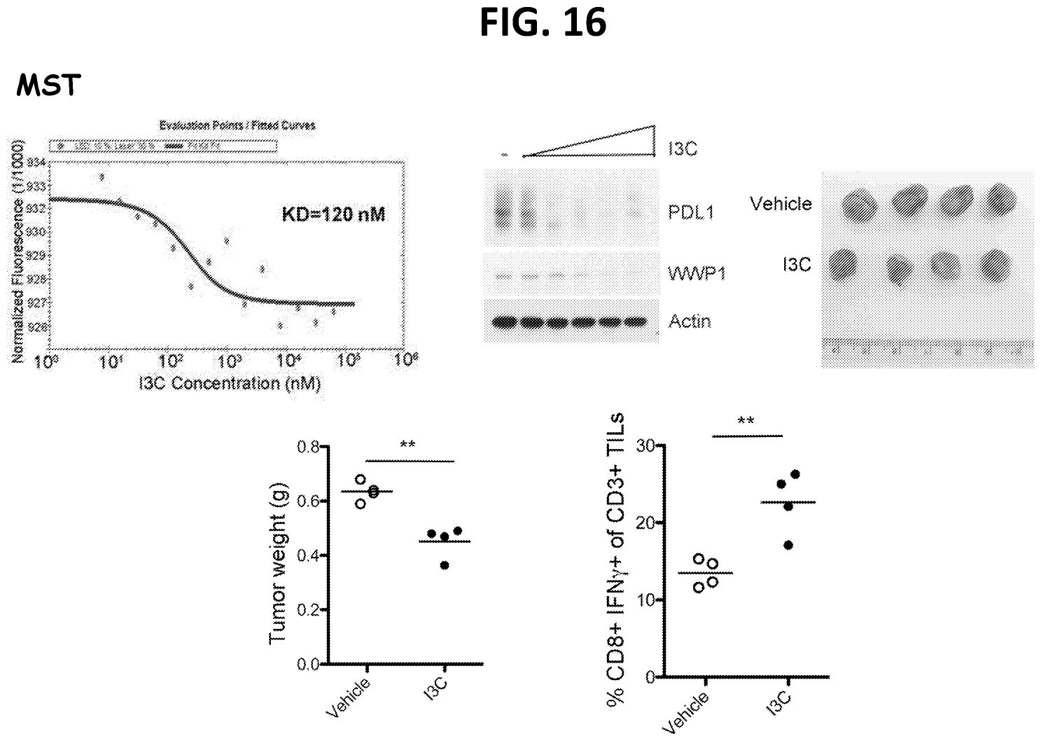

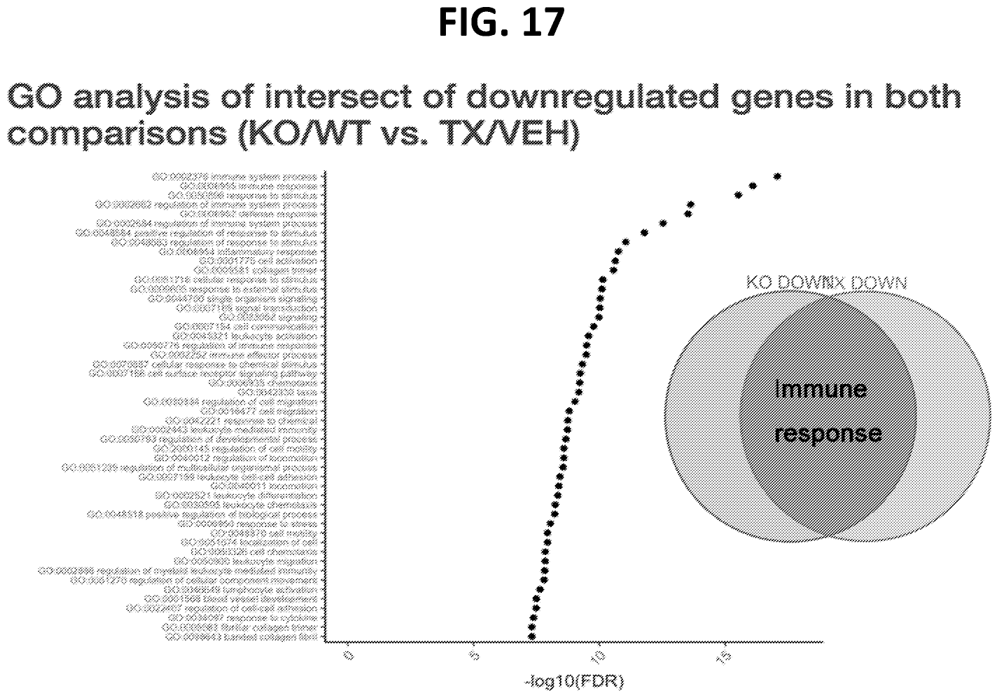

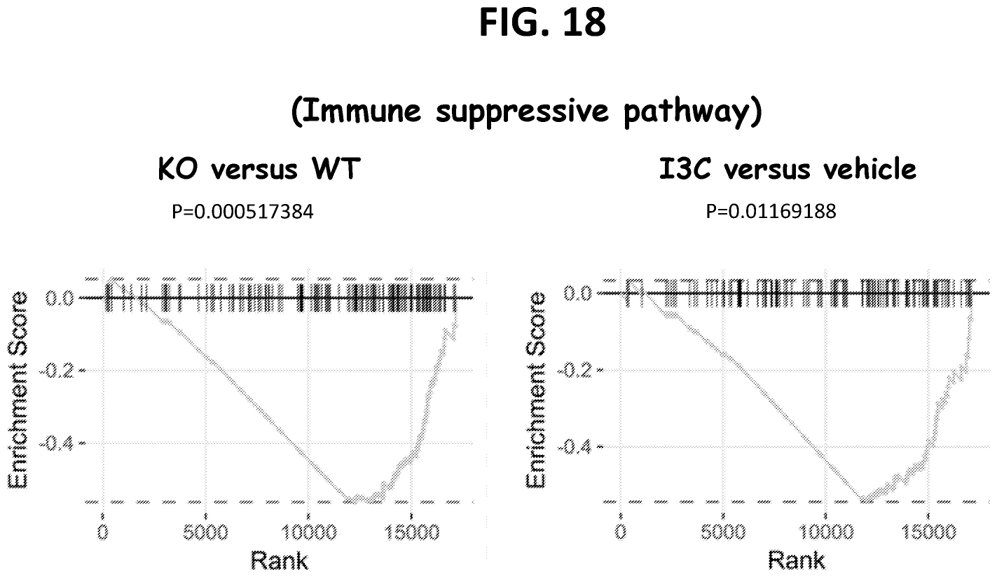

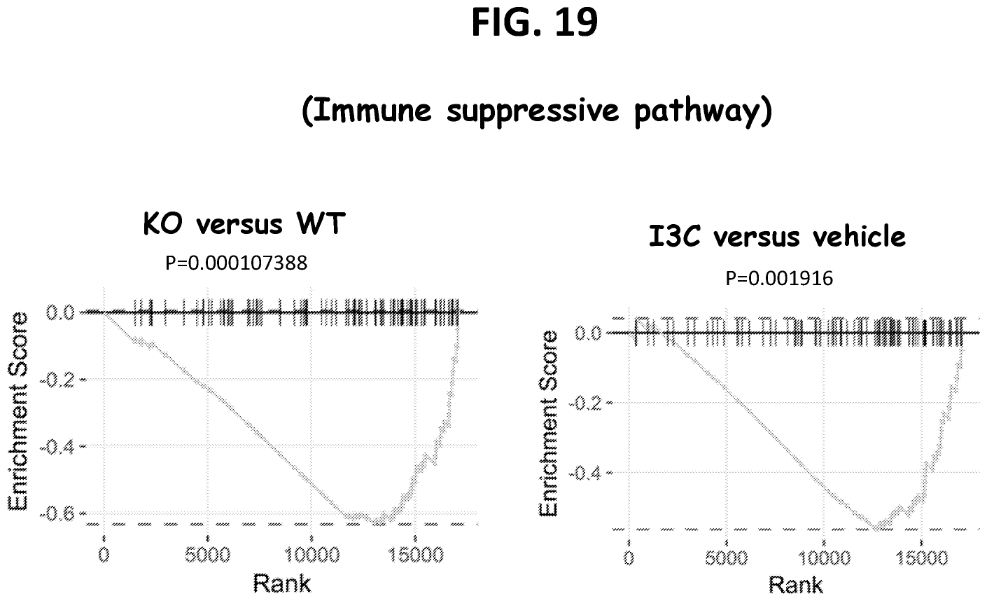

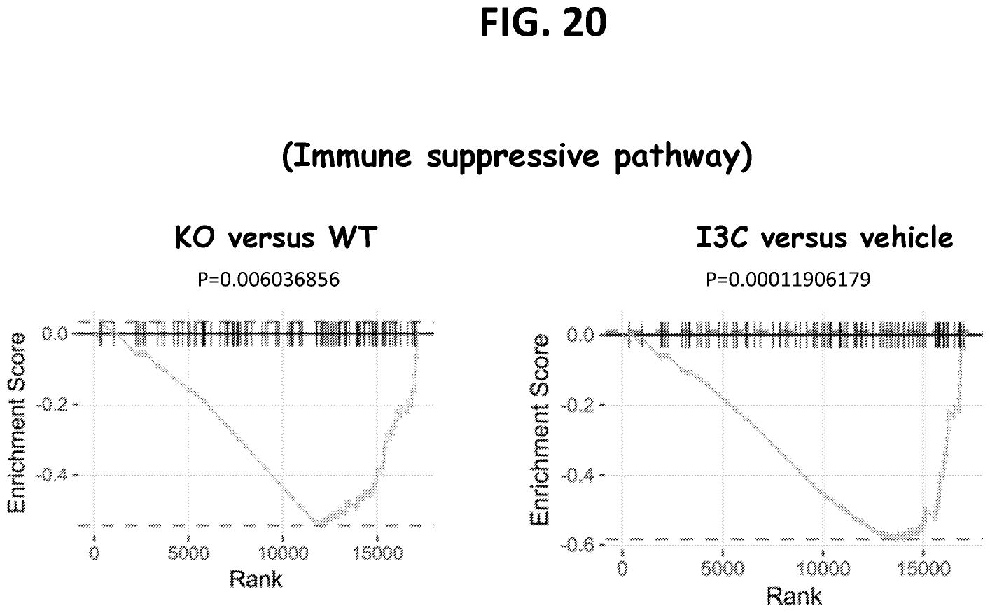

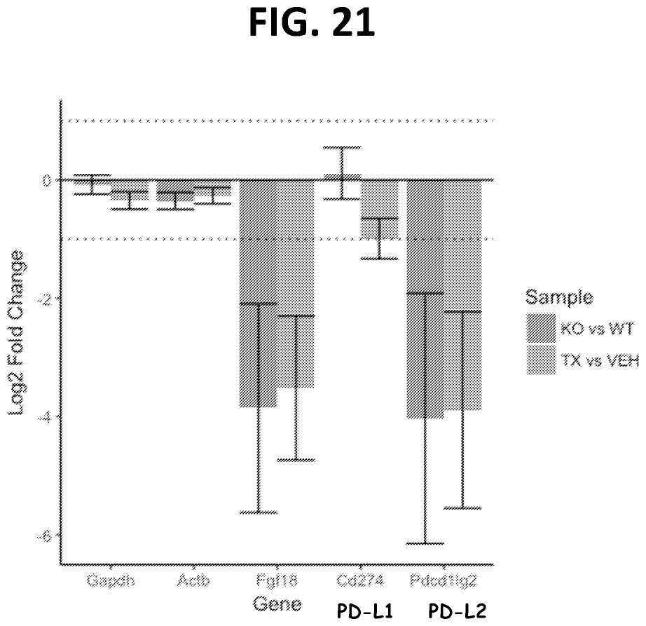

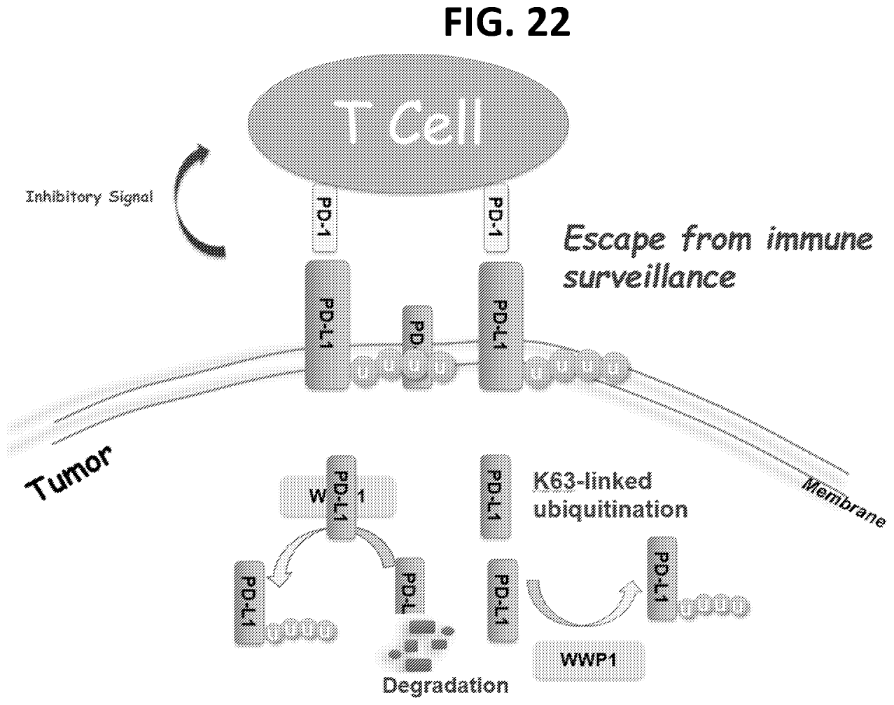

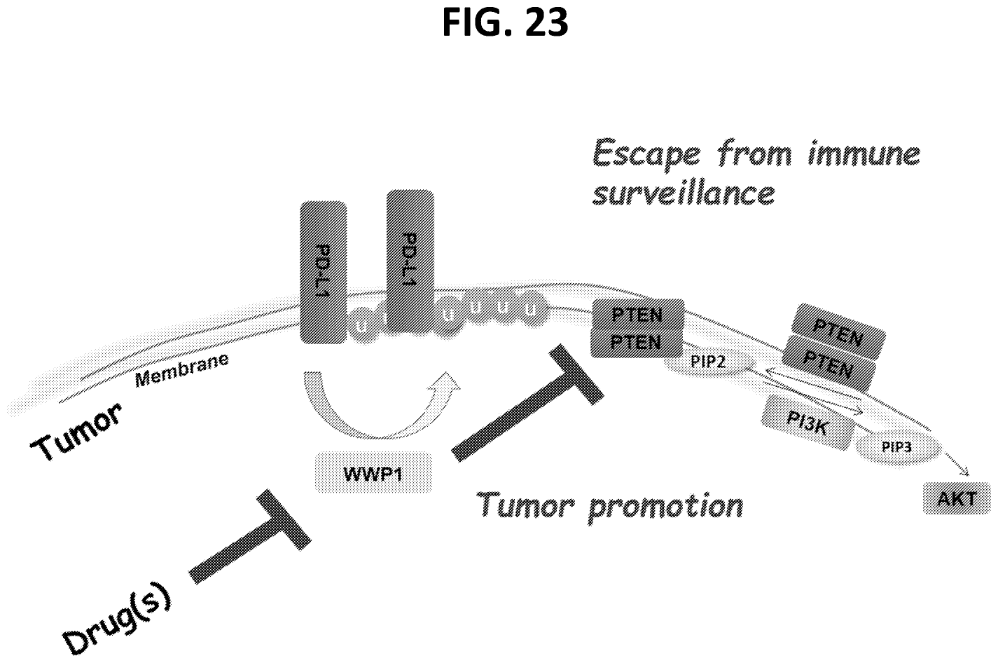

WWP1 specifically interacts with PD-L1 (FIG. 1). WWP1 triggers PD-L1 ubiquitination in a catalytic dependent manner (FIG. 2). WWP1 triggers PD-L1 K63-linked poly-ubiquitination (FIG. 3). There is a positive correlation of WWP1 and PD-L1 protein levels in triple negative breast cancer (TNBC) as shown by immunoblot (FIG. 4). Depletion of WWP1 by shRNAs reduced PD-L1 expression in triple negative breast cancer (FIG. 5), and in multiple human and mouse cancer cells (FIGS. 6A to 6D), including DLD1 cells, which is a colorectal adenocarcinoma cell line, CT26, which is a colon carcinoma cell line, HCT116, which is a human colon carcinoma cell line, and DU145, which is a human prostate cancer cell line (FIG. 7). Knockdown using shRNAs and CRISPR-mediated knockout (KO) of WWP1 robustly enhanced PD-L1 turnover and reduced its protein levels in multiple human and mice cancer cell lines (FIGS. 8A and 8B). Depletion of WWP1 reduces PD-L1 protein stability (FIG. 9). Wwp1 stabilizes PD-L1. WWP1 stabilizes PD-L1 in a catalytic dependent manner (FIGS. 10 and 11). Treatment with proteasome inhibitor MG132 restored PD-L1 protein levels in WWP1 knock-out cells (FIG. 12). WWP1-mediated PD-L1 downregulation is executed predominantly through proteasome but not autophagic pathways (FIG. 13). WWP1-mediated PD-L1 K63-Ub(n) competes for K48-Ub(n) (FIG. 14). WWP1-mediated PD-L1 degradation induced active CD8 T cell infiltration in tumors (syngenic mice) (FIG. 15). WWP1 inhibition by indole-3-carbinol (I3C), a NEDD-4-1 inhibitor, triggers active CD8 T cell tumor infiltration (syngenic mice) (FIG. 16). FIG. 17-20 show genes and pathways down regulated with WWP1 inhibition. FIG. 21 shows that inactivation of WWP1 did not affect PD-L1 mRNA levels. Increased T cell infiltration is important for an anti-tumor immune response. RNA-seq analysis reveals down-regulation of multiple immune suppressive pathway (JAK-STAT, NF-kB, and TNFα pathway) in Wwp1 KO or I3C treated Hi-Myc mice. Enhancing an anti-tumor immune response by inhibiting levels or activity of WWP1 and/or PD-L1, increased levels of tumor-infiltrating leukocytes and down regulated immune suppressive pathways, thereby treating cancer (FIGS. 22 and 23).

### OTHER EMBODIMENTS

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

This application may be related to PCT/US17/49098, which is incorporated herein in its entirety. All patents and publications mentioned in this specification are herein incorporated by reference to the same extent as if each independent patent and publication was specifically and individually indicated to be incorporated by reference.

1. A method of treating cancer in a selected subject, the method comprising administering to the subject an effective amount of an agent that inhibits the expression or activity of WWP1 and an anti-PD-1 and/or anti-PD-L1 antibody.

2. A method of treating cancer in a subject, the method comprising administering to the subject an effective amount of an agent that inhibits the expression or activity of WWP1 and an anti-PD-1 and/or anti-PD-L1 antibody.

3. A method of inhibiting the survival or proliferation of a neoplastic cell, the method comprising contacting the cell with an effective amount of an agent that inhibits the expression or activity of WWP1 and an anti-PD-1 and/or anti-PD-L1 antibody. thereby inhibiting the survival or proliferation of the neoplastic cell.

4. The method of claim 1, further comprising administering to the subject or the cell a NEDD4-1 inhibitor.

5. The method of claim 1, wherein the neoplastic cell is a mammalian cell.

6. The method of claim 5, wherein the mammalian cell is a murine, rat, or human cell.

7. The method of claim 5, wherein the cell is in vitro or in vivo.

8. The method of claim 3, wherein the neoplastic cell is a prostate cancer, breast cancer, or colorectal cancer cell.

9. The method of claim 1, wherein the subject has prostate cancer, breast cancer, or colorectal cancer.

10. The method of claim 1, wherein the agent is a polypeptide, polynucleotide, or a small molecule.

11. The method of claim 10, wherein the polynucleotide is an inhibitory nucleic acid molecule that inhibits the expression of WWP1.

12. The method of claim 11, wherein the inhibitory nucleic acid molecule is an antisense molecule, siRNA, or shRNA.

13. The method of claim 10, wherein the agent is selected from the group consisting of: 4-(4-chlorobenzoyl) piperazin-1-yl) (4-(phonoxymethyl) phenyl) methanone, and indole-3-carbinol.


SEQUENCE LISTING (6 sequences)

SEQ ID NO: 1 - protein, 403 aa, Homo sapiens [sequence omitted]

SEQ ID NO: 2 - DNA, 8718 bp, Homo sapiens
cctcccctcgcccggcgcggtcccgtccgcctctcgctcgcctcccgcctcccctcggtcttccgaggcgcccgggctcccggcgcggcggcggagggggcgggcaggccggcgggcggtgatgtggcgggactctttatgcgctgcggcaggatacgcgctcggcgctgggacgcgactgcgctcagttctctcctctcggaagctgcagccatgatggaagtttgagagttgagccgctgtgaggcgaggccgggctcaggcgagggagatgagagacggcggcggccgcggcccggagcccctctcagcgcctgtgagcagccgcgggggcagcgccctcggggagccggccggcctgcggcggcggcagcggcggcgtttctcgcctcctcttcgtcttttctaaccgtgcagcctcttcctcggcttctcctgaaagggaaggtggaagccgtgggctcgggcgggagccggctgaggcgcggcggcggcggcggcacctcccgctcctggagcgggggggagaagcggcggcggcggcggccgcggcggctgcagctccagggagggggtctgagtcgcctgtcaccatttccagggctgggaacgccggagagttggtctctccccttctactgcctccaacacggcggcggcggcggcggcacatccagggacccgggccggttttaaacctcccgtccgccgccgccgcaccccccgtggcccgggctccggaggccgccggcggaggcagccgttcggaggattattcgtcttctccccattccgctgccgccgctgccaggcctctggctgctgaggagaagcaggcccagtcgctgcaaccatccagcagccgccgcagcagccattacccggctgcggtccagagccaagcggcggcagagcgaggggcatcagctaccgccaagtccagagccatttccatcctgcagaagaagccccgccaccagcagcttctgccatctctctcctcctttttcttcagccacaggctcccagacatgacagccatcatcaaagagatcgttagcagaaacaaaaggagatatcaagaggatggattcgacttagacttgacctatatttatccaaacattattgctatgggatttcctgcagaaagacttgaaggcgtatacaggaacaatattgatgatgtagtaaggtttttggattcaaagcataaaaaccattacaagatatacaatctttgtgctgaaagacattatgacaccgccaaatttaattgcagagttgcacaatatccttttgaagaccataacccaccacagctagaacttatcaaacccttttgtgaagatcttgaccaatggctaagtgaagatgacaatcatgttgcagcaattcactgtaaagctggaaagggacgaactggtgtaatgatatgtgcatatttattacatcggggcaaatttttaaaggcacaagaggccctagatttctatggggaagtaaggaccagagacaaaaagggagtaactattcccagtcagaggcgctatgtgtattattatagctacctgttaaagaatcatctggattatagaccagtggcactgttgtttcacaagatgatgtttgaaactattccaatgttcagtggcggaacttgcaatcctcagtttgtggtctgccagctaaaggtgaagatatattcctccaattcaggacccacacgacgggaagacaagttcatgtactttgagttccctcagccgttacctgtgtgtggtgatatcaaagtagagttcttccacaaacagaacaagatgctaaaaaaggacaaaatgtttcacttttgggtaaatacattcttcataccaggaccagaggaaacctcagaaaaagtagaaaatggaagtctatgtgatcaagaaatcgatagcatttgcagtatagagcgtgcagataatgacaaggaatatctagtacttactttaacaaaaaatgatcttgacaaagcaaataaagacaaagccaaccgatacttttctccaaattttaaggtgaagctgtacttcacaaaaacagtagaggagccgtcaaatccagaggctagcagttcaacttctgtaacaccagatgttagtgacaatgaacctgatcattatagatattctgacaccactgactctgatccagagaatgaaccttttgatgaagatcagcatacacaaattacaaaagtctgaatttttttttatcaagagggataaaacaccatgaaaataaacttgaataaactgaaaatggacctttttttttttaatggcaataggacattgtgtcagattaccagttataggaacaattctcttttcctgaccaatcttgttttaccctatacatccacagggttttgacacttgttgtccagttgaaaaaaggttgtgtagctgtgtcatgtatatacctttttgtgtcaaaaggacatttaaaattcaattaggattaataaagatggcactttcccgttttattccagttttataaaaagtggagacagactgatgtgtatacgtaggaattttttccttttgtgttctgtcaccaactgaagtggctaaagagctttgtgatatactggttcacatcctacccctttgcacttgtggcaacagataagtttgcagttggctaagagaggtttccgaagggttttgctacattctaatgcatgtattcgggttaggggaatggagggaatgctcagaaaggaaataattttatgctggactctggaccatataccatctccagctatttacacacacctttctttagcatgctacagttattaatctggacattcgaggaattggccgctgtcactgcttgttgtttgcgcatttttttttaaagcatattggtgctagaaaaggcagctaaaggaagtgaatctgtattggggtacaggaatgaaccttctgcaacatcttaagatccacaaatgaagggatataaaaataatgtcataggtaagaaacacagcaacaatgacttaaccatataaatgtggaggctatcaacaaagaatgggcttgaaacattataaaaattgacaatgatttattaaatatgttttctcaattgtaacgacttctccatctcctgtgtaatcaaggccagtgctaaaattcagatgctgttagtacctacatcagtcaacaacttacacttattttactagttttcaatcataatacctgctgtggatgcttcatgtgctgcctgcaagcttcttttttctcattaaatataaaatattttgtaatgctgcacagaaattttcaatttgagattctacagtaagcgttttttttctttgaagatttatgatgcacttattcaatagctgtcagccgttccacccttttgaccttacacattctattacaatgaattttgcagttttgcacattttttaaatgtcattaactgttagggaattttacttgaatactgaatacatataatgtttatattaaaaaggacatttgtgttaaaaaggaaattagagttgcagtaaactttcaatgctgcacacaaaaaaaagacatttgatttttcagtagaaattgtcctacatgtgctttattgatttgctattgaaagaatagggtttttttttttttttttttttttttttttaaatgtgcagtgttgaatcatttcttcatagtgctcccccgagttgggactagggcttcaatttcacttcttaaaaaaaatcatcatatatttgatatgcccagactgcatacgattttaagcggagtacaactactattgtaaagctaatgtgaagatattattaaaaaggtttttttttccagaaatttggtgtcttcaaattataccttcaccttgacatttgaatatccagccattttgtttcttaatggtataaaattccattttcaataacttattggtgctgaaattgttcactagctgtggtctgacctagttaatttacaaatacagattgaataggacctactagagcagcatttatagagtttgatggcaaatagattaggcagaacttcatctaaaatattcttagtaaataatgttgacacgttttccataccttgtcagtttcattcaacaatttttaaatttttaacaaagctcttaggatttacacatttatatttaaacattgatatatagagtattgattgattgctcataagttaaattggtaaagttagagacaactattctaacacctcaccattgaaatttatatgccaccttgtctttcataaaagctgaaaattgttacctaaaatgaaaatcaacttcatgttttgaagatagttataaatattgttctttgttacaatttcgggcaccgcatattaaaacgtaactttattgttccaatatgtaacatggagggccaggtcataaataatgacattataatgggcttttgcactgttattatttttcctttggaatgtgaaggtctgaatgagggttttgattttgaatgtttcaatgtttttgagaagccttgcttacattttatggtgtagtcattggaaatggaaaaatggcattatatatattatatatataaatatatattatacatactctccttactttatttcagttaccatccccatagaatttgacaagaattgctatgactgaaaggttttcgagtcctaattaaaactttatttatggcagtattcataattagcctgaaatgcattctgtaggtaatctctgagtttctggaatattttcttagactttttggatgtgcagcagcttacatgtctgaagttacttgaaggcatcacttttaagaaagcttacagttgggccctgtaccatcccaagtcctttgtagctcctcttgaacatgtttgccatacttttaaaagggtagttgaataaatagcatcaccattctttgctgtggcacaggttataaacttaagtggagtttaccggcagcatcaaatgtttcagctttaaaaaataaaagtagggtacaagtttaatgtttagttctagaaattttgtgcaatatgttcataacgatggctgtggttgccacaaagtgcctcgtttacctttaaatactgttaatgtgtcatgcatgcagatggaaggggtggaactgtgcactaaagtgggggctttaactgtagtatttggcagagttgccttctacctgccagttcaaaagttcaacctgttttcatatagaatatatatactaaaaaatttcagtctgttaaacagccttactctgattcagcctcttcagatactcttgtgctgtgcagcagtggctctgtgtgtaaatgctatgcactgaggatacacaaaaataccaatatgatgtgtacaggataatgcctcatcccaatcagatgtccatttgttattgtgtttgttaacaaccctttatctcttagtgttataaactccacttaaaactgattaaagtctcattcttgtcattgtgtgggtgttttattaaatgagagtttataattcaaattgcttaagtccattgaagttttaattaatgggcagccaaatgtgaatacaaagttttcagtttttttttttcctgctgtccttcaaagcctactgtttaaaaaaaaaaaaaaaaaaaaacatggcctgagagtagagtatctgtctactcatgtttaattaaggaaaaacacttatttttagggctttagtcatcacttcataaattgtataagcacattaaatagcgttctagtcctgaaaaagtccaagattcttagaaaattgtgcatatttttattatgacagatgtttgaagataattccccagaatggatttgatactttagatttcaattttgtggcttttgtctattattctgtactctgccatcagcatatggaaagcttcatttactcatcatgacttgtgccatataaaaattgatatttcggaatagtctaaaggactttttgtacttgaatttaatcatgttgtttctaatattcttaaaagcttgaagactaaagcatatcctttcaacaaagcatagtaaggtaataagaaagtgtagtttgtacaagtgttaaaaaaataaagtagacaatgttacagtgggacttattatttcaagtttacattttctccatgtaattttttaaaaagtaaatgaaaaaatgtgcaataatgtaaaatatgaagtgtatgtgtacacacattttatttttcggtatcttgggtatacgtatggttgaaaactatactggagtctaaaagtattctaatttataagaagacattttggtgatgtttgaaaaatagaaatgtgctagttttgtttttatatcatgtcctttgtacgttgtaatatgagctggcttggttcagtaaatgccatcaccatttccattgagaatttaaaactcaccagtgtttaatatgcaggcttccaaaggcttatgaaaaaaatcaagacccttaaatctagttaatttgctgctaacatgaaactctttggttcttttatttttgccagataattagacacacatctaaagcttagtcttaaatggcttaagtgtagctattgattagtgctgttgctagttcagaaagaaatgtttgtgaatggaaacaagaatattcagtccaaactgttgtaaggacagtacctgaaaaccaggaaacaggataatggaaaaagtcttttaaagatgaaatgttggagccaactttcttatagaattaattgtatgtggctatagaaagcctaatgattgttgcttatttttgagagcatattattcttttatgaccataatcttgctgtttttccatcttccaaaagatcttccttctaatatgtatatcagaatgtgggtagccagtcagacaaattcatattggttggtagctttaaaaagtttgtaatgtgaagacaggaaaggacaaaatagtttgctttggtggtagtactctggttgttaagctaggtattttgagactacttccccatcacaacaacaataaaataatcactcataatcctatcacctggagacatagccatcgttaatatgttagtgactatacaatcatgttttcttctgtatatccatgtatattctttaaaaatgaaatttatactgtacctgatctcaaagctttttagcttagtatatctgtcatgaatttgtaggatgttccattgcatcagaaaacggacagtgatttgattactttctaatgccacagatgcagattacatgtagttattgagaatcctttcgaattcagtggcttaatcatgaatgtctaaatattgttgacattaggatgatacatgtaaattaaagttacatttgtttagcatagacaagcttaacattgtagatgtttctcttcaaaaatcatcttaaacatttgcatttggaattgtgttaaatagaatgtgtgaaacactgtattagtaaacttcatcacctttctacttccttatagtttgaacttttcagtttttgtagttcccaaacagttgctcaatttagagcaaattaatttaacacctgccaaaaaaaggctgctgttggcttatcagttgtctttaaattcaaatgctcatgtgacttttatcacatcaaaaaatatttcattaatgattcacctttagctctgaaaattaccgcgtttagtaattatagtgggcttataaaaacatgcaactctttttgatagttatttgagaattttggtgaaaaatatttagctgagggcagtatagaacttataaaccaatatattgatatttttaaaacatttttacatataagtaaactgccatctttgagcataactacatttaaaaataaagctgcatatttttaaatcaagtgtttaacaagaatttatattttttattttttaaaattaaaaataatttatatttcctctgttgcatgaggattctcatctgtgcttataatggttagagattttatttgtgtggaatgaagtgaggcttgtagtcatggttctagtgtttcagtttgccaagtctgtttactgcagtgaaattcatcaaatgtttcagtgtggttttctgtagcctatcatttactggctatttttttatgtacacctttaggattttctgcctactctatccagttgtccaaatgatatcctacattttacaaatgccctttcagtttctattttctttttccattaaattgccctcatgtcctaatgtgcagtttgtaagtgtgtgtgtgtgtgtctgtgtgtgtgtgaatttgattttcaagagtgctagacttccaatttgagagattaaataatttaattcaggcaaacatttttcattggaatttcacagttcattgtaatgaaaatgttaatcctggatgacctttgacatacagtaatgaatcttggatattaatgaatttgttagtagcatcttgatgtgtgttttaatgagttattttcaaagttgtgcattaaaccaaagttggcatactggaagtgtttatatcaagttccatttggctactgatggacaaaaaatagaaatgccttcctatggagagtatttttcctttaaaaaattaaaaaggttaattattttgactaaaaaaaaaaaaaaaaaa

SEQ ID NO: 3 - protein, 900 aa, Homo sapiens
MATCAVEVFGLLEDEENSRIVRVRVIAGIGLAKKDILGASDPYVRVTLYDPMNGVLTSVQTKTIKKSLNPKWNEEILFRVHPQQHRLLFEVFDENRLTRDDFLGQVDVPLYPLPTENPRLERPYTFKDFVLHPRSHKSRVKGYLRLKMTYLPKTSGSEDDNAEQAEELEPGWVVLDQPDAACHLQQQQEPSPLPPGWEERQDILGRTYYVNHESRRTQWKRPTPQDNLTDAENGNIQLQAQRAFTTRRQISEETESVDNRESSENWEIIREDEATMYSNQAFPSPPPSSNLDVPTHLAEELNARLTIFGNSAVSQPASSSNHSSRRGSLQAYTFEEQPTLPVLLPTSSGLPPGWEEKQDERGRSYYVDHNSRTTTWTKPTVQATVETSQLTSSQSSAGPQSQASTSDSGQQVTQPSEIEQGFLPKGWEVRHAPNGRPFFIDHNTKTTTWEDPRLKIPAHLRGKTSLDTSNDLGPLPPGWEERTHTDGRIFYINHNIKRTQWEDPRLENVAITGPAVPYSRDYKRKYEFFRRKLKKQNDIPNKFEMKLRRATVLEDSYRRIMGVKRADFLKARLWIEFDGEKGLDYGGVAREWFFLISKEMFNPYYGLFEYSATDNYTLQINPNSGLCNEDHLSYFKFIGRVAGMAVYHGKLLDGFFIRPFYKMMLHKPITLHDMESVDSEYYNSLRWILENDPTELDLRFIIDEELFGQTHQHELKNGGSEIVVTNKNKKEYIYLVIQWRFVNRIQKQMAAFKEGFFELIPQDLIKIFDENELELLMCGLGDVDVNDWREHTKYKNGYSANHQVIQWFWKAVLMMDSEKRIRLLQFVTGTSRVPMNGFAELYGSNGPQSFTVEQWGTPEKLPRAHTCFNRLDLPPYESFEELWDKLQMAIENTQGFDGVD

SEQ ID NO: 4 - DNA, 7240 bp, Homo sapiens
gctgcagctgagctcagcttaaaggtcgctggaaatcaagtgctttgtaagtgaaggcattttgactacaagcgattcaagacattttggaaacgtcagtctctggaatgggtcgtctttttctttctatgatatctggtatctaatgttttacttaggataaagactactacttaatagctcttttaggcaccacaaatgattttttcttttgatgcagtgcagctgcttctagcctgtctacggacagagatctttgttatgactttcagcccttaacatgtttggaaatgagaacaaatggcacaaagcttacgattgcactttgcagccagaagaagcaatacttaccctttgtcagaaacctccggagatgacttggatagccatgttcacatgtgcttcaaaagaccaacacggatttcaacgtctaacgttgttcaaatgaagctgactcccagacagactgcactagctccgttaataaaggaaaacgttcagtctcaagaaagatcatctgttccctcatctgaaaatgttaataaaaagagcagctgtctacagatttcactacagccaacaaggtacagtggatatcttcagtctagcaatgtcttagctgatagtgatgatgcttcgtttacttgtatcttgaaggatggtatttacagtagtgctgtggtcgataatgaattgaatgctgtgaatgatggtcaccttgtaagcagtccagccatttgtagtggtagccttagtaacttttcaaccagtgataatgggtcttacagcagcaacggtagtgattttgggtcatgtgcaagtatcacaagtggaggttcatatactaacagtgtcatcagtgacagtagtagttatacttttccaccaagtgatgatacttttttgggtggaaacttaccttctgacagcacctccaatagaagtgtgccaaacaggaatactactccttgtgaaattttttcaagaagtacaagtacagatccttttgtccaggatgacttggaacatggattagagattatgaaattgccagtgagcaggaacacaaaaattccactaaaacgttactcctccttagtcatttttcctaggagtccttcaactacccgaccgacttctccaacaagtctgtgtactcttctgagcaaaggatcctatcaaacttcacaccagtttattatttctcctagtgaaattgcacataatgaggatggcactagtgctaaaggatttctttcaacagctgtcaatggacttcggttatctaaaacaatttgtacccccggagaagtaagagacatacggccgcttcacaggaagggctcgttacagaagaaaattgttctttcgaataatactcccagacagactgtctgtgaaaagtcatctgaaggatattcttgtgtttcagtgcatttcacccaacgaaaagcagctacattagactgtgaaacaacaaatggtgattgtaaaccagaaatgtcagaaattaagcttaattctgattcagagtatattaagctcatgcataggacatctgcatgtttgccatcctcccaaaatgtagattgtcaaataaatatcaatggagaattggaaagaccacattcacagatgaacaaaaaccatggtattttacgaagaagtatttcattgggaggagcttatccaaatatttcctgtctatccagccttaagcacaattgttctaaagggggaccatctcagttactcataaagtttgcatctggaaatgaaggtaaagtggataatttatcaagagacagcaacagagattgcacaaatgaactgtctaattcttgcaagacaagagatgatttcctaggtcaagtggatgttccactttatccattaccgacagaaaatccaagattggagagaccatatacatttaaggattttgttcttcatccaagaagtcacaaatcaagagttaaaggttatctgagactaaaaatgacttatttacctaaaaccagtggctcagaagatgataatgcagaacaggctgaggaattagagcctggctgggttgttttggaccaaccagatgctgcttgccatttgcagcaacaacaagaaccttctcctctacctccagggtgggaagagaggcaggatatccttggaaggacctattatgtaaaccatgaatctagaagaacacagtggaaaagaccaacccctcaggacaacctaacagatgctgagaatggcaacattcaactgcaagcacaacgtgcatttaccaccaggcggcagatatccgaggaaacagaaagtgttgacaaccgagagtcttccgagaactgggaaattataagagaagatgaagccaccatgtatagcaaccaggccttcccatcacctccaccgtcaagtaacttggatgttccaactcatcttgcagaagaattgaatgccagactcaccatttttggaaattcagccgtgagccagccagcatcgagctcaaatcattccagcagaagaggcagcttacaagcctatacttttgaggaacaacctacacttcctgtgcttttgcctacttcatctggattaccaccaggttgggaagaaaaacaagatgaaagaggaagatcatattatgtagatcacaattccagaacgactacttggacaaagcccactgtacaggccacagtggagaccagtcagctgacctcaagccagagttctgcaggccctcaatcacaagcctccaccagtgattcaggccagcaggtgacccagccatctgaaattgagcaaggattccttcctaaaggctgggaagtccggcatgcaccaaatgggaggcctttctttattgaccacaacactaaaaccaccacctgggaagatccaagattgaaaattccagcccatctgagaggaaagacatcacttgatacttccaatgatctagggcctttacctccaggatgggaagagagaactcacacagatggaagaatcttctacataaatcacaatataaaaagaacacaatgggaagatcctcggttggagaatgtagcaataactggaccagcagtgccctactccagggattacaaaagaaagtatgagttcttccgaagaaagttgaagaagcagaatgacattccaaacaaatttgaaatgaaacttcgccgagcaactgttcttgaagactcttaccggagaattatgggtgtcaagagagcagacttcctgaaggctcgactgtggattgagtttgatggtgaaaagggattggattatggaggagttgccagagaatggttcttcctgatctcaaaggaaatgtttaacccttattatgggttgtttgaatattctgctacggacaattataccctacagataaatccaaactctggattgtgtaacgaagatcacctctcttacttcaagtttattggtcgggtagctggaatggcagtttatcatggcaaactgttggatggttttttcatccgcccattttacaagatgatgcttcacaaaccaataacccttcatgatatggaatctgtggatagtgaatattacaattccctaagatggattcttgaaaatgacccaacagaattggacctcaggtttatcatagatgaagaactttttggacagacacatcaacatgagctgaaaaatggtggatcagaaatagttgtcaccaataagaacaaaaaggaatatatttatcttgtaatacaatggcgatttgtaaaccgaatccagaagcaaatggctgcttttaaagagggattctttgaactaataccacaggatctcatcaaaatttttgatgaaaatgaactagagcttcttatgtgtggactgggagatgttgatgtgaatgactggagggaacatacaaagtataaaaatggctacagtgcaaatcatcaggttatacagtggttttggaaggctgttttaatgatggattcagaaaaaagaataagattacttcagtttgtcactggcacatctcgggtgcctatgaatggatttgctgaactatacggttcaaatggaccacagtcatttacagttgaacagtggggtactcctgaaaagctgccaagagctcatacctgttttaatcgcctggacttgccaccttatgaatcatttgaagaattatgggataaacttcagatggcaattgaaaacacccagggctttgatggagttgattagattacaaataacaatctgtagtgtttttactgccatagttttataaccaaaatcttgacttaaaattttccggggaactactaaaatgtggccactgagtcttcccagatcttgaagaaaatcatataaaaagcatttgaagaaatagtacgacaacttatttttaatcacttttaaataatgtgttgcatttacacagttgtttcatgctgtctttagagttaggtgcctgcctaaagccaggcaccaccacacctggctttagagttcacacaataggatataagtcctgtatgacttaaatagtgaattttgtccttaacatttacctcttgtatagtatctgccaggcagttttttcttaaactactgagatgataactgtgaaatatttgtgatacgtgtcatgtgtgaaaagtttgatgcattttgagatggaaaactgaaatttggaaaaagaaatactttactattgagtaaactacaatatatttagtgctactcgcagctatttattattttgtagacctgccttatgcaccttactgcctagatttttgggaaaaaactttggaaagtgtgttacctatatttctagccaactaactcacagaaaaactgtttacttcttcactttcgaagtatttggcttttgttaatatgcagttttactaaacagatggttcataagacatgtgaagcaaattcatatttgcaatgggtaaaaagtattaaagcctttctcttgcctgcatatcctattgaccattggtatagtcatcactttttcatattttagtgtagttagaagaattccttcttcaaacattaaagatccacaaagcagtatttctaaatatgccttgaagaactaaatgaagtgtatagcacttgcctttactagatattctttacacttgtacaattatgtagtaaatgtatgtttacagggtttatcatgtttacagattaagctaatttctgtagtcgcatttttatatttttagtatcactctagtaaaaaaaccaaataatttgtttaaaataaccaaagagttgttataatgcataatttgtattaaatttattactatttcttatgccttttaaaatactgtttactatgaagacaatgtttttaattacaaatccagaattctgtaggcaaaatgctacagttcatatcttcctttaaccaaactgaagtacataaagaccatgtacatgtattcatcaaacgtttattgaatgctgcgtgcctagcgctgtgctatgctctggggtaagagttgtcagcttcagagaagctgagtcctgatcctcaaggaacttgcaaatgtgtctatgaatttgtaaaacaatcaaaagtaggcgtaagcagaataaggcaaaagggaaagtgttctaggttccagcacacctgcaaagataaagtgtgccaagactgtatttatatttcatacttatattgtttcatccttatattggaatgattatatagaaaatgctcttaaaaagattaaacctatttctcagtatggtatcttggtgatttaggaataattgtaaatatatgttacgaatcttcttaaatatatatatatacacaccctgtgagaactgtaaaaagtacctctggttcttggtttaagtttgttggggtataacatgatgagtactcattagcacctgatagaaatctgaaatgtgacagtagcaaaaccactttctactttccaaacaccacagcatcagcatggtttaggggaagcaattcacagattaatgtaccctgcgttttgtcttcaccattgtcaaccagcagtcaaggatgagcaccaggtatagtcctggtttgatatctcaccagccatgtgaactgaagcagcttacttaacctctctggattcatatttcttcatctgtcaaatgagagtaatcatgcccaccctcattgtcatgccagattgtcagaagttccagacagatgagaaagcaaaaagtactttgtaaagtgttatacaatcagaaggtgctttgtgctgaccgtcagattgtgtcaagtcaggtgtgcaaattgaccacactcccaaagaatcatttaaaagtaaagtgacttctaagagacaaggaagtaggaacatttctgatattaaatatataaataatatggatacacatgtgtatatctgtatatggatcttaccatcataattcattttcttcatatcagcaaagtaatagtaatgtgggccaatgcattttggaaatgtcctcattatgtagaatggaatgtgaaaattatttttgttaaaacaggattttgccacaattatttaaatattatgtttgtgaactatctaagcatgagaaaatacaaagctttctttgtatctggctgatactcatttggatgattctgtgactcatagaacatgatgttaaatgagaccagactcaactgccaccagtccccagctgcagaccttcatccccttcatctcccactaggggctcgtggtctggaagaaacatctatcaagcaacacagccccttatcccagatagaaaagtgtctcaaatgcattctactgcgggacagtcagtaggatcattttcataaagcaaggacatcatatgtttctagaaattacaagcatataactttagcctacaatctcttattaaaaatttttaacaaaattgtattacaaatgcatttcatcagaactcaaatttaaatggtgtttgttttgggtttttatttataatgctgaagttattccatataagtatcaagttaaacacaattcattttgattataaactatttgactttttaaaatcttctgacacagtaaatatatatatcaagattgatgtatcaaaatttattgcacactttaaagtgtaaaatcattttttaaaatcttgaatccacaaataaagttctattctgattttaaaaaacaaaacaaaa

SEQ ID NO: 5 - protein, 922 aa, Homo sapiens
MATASPRSDTSNNHSGRLQLQVTVSSAKLKRKKNWFGTAIYTEVVVDGEITKTAKSSSSSNPKWDEQLTVNVTPQTTLEFQVWSHRTLKADALLGKATIDLKQALLIHNRKLERVKEQLKLSLENKNGIAQTGELTVVLDGLVIEQENITNCSSSPTIEIQENGDALHENGEPSARTTARLAVEGTNGIDNHVPTSTLVQNSCCSYVVNGDNTPSSPSQVAARPKNTPAPKPLASEPADDTVNGESSSFAPTDNASVTGTPVVSEENALSPNCTSTTVEDPPVQEILTSSENNECIPSTSAELESEARSILEPDTSNSRSSSAFEAAKSRQPDGCMDPVRQQSGNANTETLPSGWEQRKDPHGRTYYVDHNTRTTTWERPQPLPPGWERRVDDRRRVYYVDHNTRTTTWQRPTMESVRNFEQWQSQRNQLQGAMQQFNQRYLYSASMLAAENDPYGPLPPGWEKRVDSTDRVYFVNHNTKTTQWEDPRTQGLQNEEPLPEGWEIRYTREGVRYFVDHNTRTTTFKDPRNGKSSVTKGGPQIAYERGFRWKLAHFRYLCQSNALPSHVKINVSRQTLFEDSFQQIMALKPYDLRRRLYVIFRGEEGLDYGGLAREWFFLLSHEVLNPMYCLFEYAGKNNYCLQINPASTINPDHLSYFCFIGRFIAMALFHGKFIDTGFSLPFYKRMLSKKLTIKDLESIDTEFYNSLIWIRDNNIEECGLEMYFSVDMEILGKVTSHDLKLGGSNILVTEENKDEYIGLMTEWRFSRGVQEQTKAFLDGFNEVVPLQWLQYFDEKELEVMLCGMQEVDLADWQRNTVYRHYTRNSKQIIWFWQFVKETDNEVRMRLLQFVTGTCRLPLGGFAELMGSNGPQKFCIEKVGKDTWLPRSHTCFNRLDLPPYKSYEQLKEKLLFAIEETEGFGQE

SEQ ID NO: 6 - DNA, 4120 bp, Homo sapiens
ggctgctggcggcctgggctgccggggccgacgcctgggtggctgctgccgccgcgcctgctgcgagatggcgatcttgggcgcggaagggtgagggcgcccgccgcaggaggaggtgccgctgccgtggccgcccggctgccgggagccgacagcttcgcgccggggttgtctcctcacagactatgagctccttgaaagagggaatcgtgtcttactcatctttgtatccccagtgtctagcagttcctgatacatagttttagctgaattttgggacatggccactgcttcaccaaggtctgatactagtaataaccacagtggaaggttgcagttacaggtaactgtttctagtgccaaacttaaaagaaaaaagaactggttcggaacagcaatatatacagaagtagttgtagatggagaaattacgaaaacagcaaaatccagtagttcttctaatccaaaatgggatgaacagctaactgtaaatgttacgccacagactacattggaatttcaagtttggagccatcgcactttaaaagcagatgctttattaggaaaagcaacgatagatttgaaacaagctctgttgatacacaatagaaaattggaaagagtgaaagaacaattaaaactttccttggaaaacaagaatggcatagcacaaactggtgaattgacagttgtgcttgatggattggtgattgagcaagaaaatataacaaactgcagctcatctccaaccatagaaatacaggaaaatggtgatgccttacatgaaaatggagagccttcagcaaggacaactgccaggttggctgttgaaggcacgaatggaatagataatcatgtacctacaagcactctagtccaaaactcatgctgctcgtatgtagttaatggagacaacacaccttcatctccgtctcaggttgctgccagacccaaaaatacaccagctccaaaaccactcgcatctgagcctgccgatgacactgttaatggagaatcatcctcatttgcaccaactgataatgcgtctgtcacgggtactccagtagtgtctgaagaaaatgccttgtctccaaattgcactagtactactgttgaagatcctccagttcaagaaatactgacttcctcagaaaacaatgaatgtattccttctaccagtgcagaattggaatctgaagctagaagtatattagagcctgacacctctaattctagaagtagttctgcttttgaagcagccaaatcaagacagccagatgggtgtatggatcctgtacggcagcagtctgggaatgccaacacagaaaccttgccatcagggtgggaacaaagaaaagatcctcatggtagaacctattatgtggatcataatactcgaactaccacatgggagagaccacaacctttacctccaggttgggaaagaagagttgatgatcgtagaagagtttattatgtggatcataacaccagaacaacaacgtggcagcggcctaccatggaatctgtccgaaattttgaacagtggcaatctcagcggaaccaattgcagggagctatgcaacagtttaaccaacgatacctctattcggcttcaatgttagctgcagaaaatgacccttatggacctttgccaccaggctgggaaaaaagagtggattcaacagacagggtttactttgtgaatcataacacaaaaacaacccagtgggaagatccaagaactcaaggcttacagaatgaagaacccctgccagaaggctgggaaattagatatactcgtgaaggtgtaaggtactttgttgatcataacacaagaacaacaacattcaaagatcctcgcaatgggaagtcatctgtaactaaaggtggtccacaaattgcttatgaacgcggctttaggtggaagcttgctcacttccgttatttgtgccagtctaatgcactacctagtcatgtaaagatcaatgtgtcccggcagacattgtttgaagattccttccaacagattatggcattaaaaccctatgacttgaggaggcgcttatatgtaatatttagaggagaagaaggacttgattatggtggcctagcgagagaatggtttttcttgctttcacatgaagttttgaacccaatgtattgcttatttgagtatgcgggcaagaacaactattgtctgcagataaatccagcatcaaccattaatccagaccatctttcatacttctgtttcattggtcgttttattgccatggcactatttcatggaaagtttatcgatactggtttctctttaccattctacaagcgtatgttaagtaaaaaacttactattaaggatttggaatctattgatactgaattttataactcccttatctggataagagataacaacattgaagaatgtggcttagaaatgtacttttctgttgacatggagattttgggaaaagttacttcacatgacctgaagttgggaggttccaatattctggtgactgaggagaacaaagatgaatatattggtttaatgacagaatggcgtttttctcgaggagtacaagaacagaccaaagctttccttgatggttttaatgaagttgttcctcttcagtggctacagtacttcgatgaaaaagaattagaggttatgttgtgtggcatgcaggaggttgacttggcagattggcagagaaatactgtttatcgacattatacaagaaacagcaagcaaatcatttggttttggcagtttgtgaaagagacagacaatgaagtaagaatgcgactattgcagttcgtcactggaacctgccgtttacctctaggaggatttgctgagctcatgggaagtaatgggcctcaaaagttttgcattgaaaaagttggcaaagacacttggttaccaagaagccatacatgttttaatcgcttggatctaccaccatataagagttatgaacaactaaaggaaaaacttctttttgcaatagaagagacagagggatttggacaagaatgaatgtggcttcttattttggaggagctcttgcatttaaataccccagccaagaaaaattgcacagatagtgtatataagctgttcattctgtacagtgaattttccgaacctctcaaagtatgttttccgttcttccacagaaatatgcaaaacagttcatccttttctactttatttattgttcccttgaaatgactgaccaggaaaaagatcatccttaaattttgaagcaagtgagagactttattaaaaatacatatatatctatataaacatatatgatagtggctctagttttatagagctccaagtgtattaaacatgacagccattcattcataaagatctggatttgctttaccttgttaatattatctaggggaaaaagtgcaaattgctccatgttcttctctcccttatgtaacatctcctgagggtgtttagttgcatggctgttcagaaaggtattaagggcttaggccaaatcttactttgagtatgttaaaaaaaaaaaaatgctgctggcttttctgaagacaggtgcttgaacttgtcagtttgttttaaataaatacaatagttgaaaatttttctctgttacatcagtaatattgttaaagtaatggatagaaccataacttacacatgaaagtcatatactagatccaatactatttagtttattatcgaaattggaaggattcattgagcagcatagaagtttgtttacatgttactttgagatgctaggtatttgtggaattaaaaagaatcaggctcttttgtactttgtttttaaatctgtgatgcttttcaaatttaattcataataaattgatgcaatttcatacttaggaacatacaaaaggtaatgtaaactctgccacttttttgtgttcaaaattttggtttttatgaagccagatggattgaagagttacataagcatttgaatgctctaatataaggctaatgattttctgttagtgtttgaatatcttcattcctctcaaattcataacagttctatttaactgaattaaataaccatatgaaaaaaaaaaaaaaaaa